(19) United States
(12) Patent Application Publication
(10) Pub. No.: US 2013/0052644 A1
(43) Pub. Date: Feb. 28, 2013

(54) Predictive Markers Useful in the Treatment of Fragile X Syndrome (FXS)

(71) Applicants: Baltazar Gomez-Mancilla, Basel (CH); Yunsheng He, Waltham, MA (US); Donald Johns, Woburn, MA (US); Joanne Meyer, Framingham, MA (US); Charles Paulding, Groton, MA (US)

(72) Inventors: Baltazar Gomez-Mancilla, Basel (CH); Yunsheng He, Waltham, MA (US); Donald Johns, Woburn, MA (US); Joanne Meyer, Framingham, MA (US); Charles Paulding, Groton, MA (US)

(21) Appl. No.: 13/695,214

(22) PCT Filed: Apr. 28, 2011
(86) PCT No.: PCT/US2011/034244

§ 371 Date: Oct. 29, 2012

Related U.S. Application Data
(60) Provisional application No. 61/330,299, filed on Apr. 30, 2010.
(60) Provisional application No. 61/379,197, filed on Sep. 1, 2010.

(51) Int. Cl. C12Q 1/68; G01N 33/566; G01N 33/53

(52) U.S. Cl. 435/6.11; 435/6.12; 435/7.1; 435/7.92; 436/501

(57) ABSTRACT

The invention is directed to the use of biomarkers to determine responsiveness of an individual with Fragile X Syndrome (FXS) to treatment with an mGluR5 antagonist.

### FIELD OF THE INVENTION

The present invention relates to a method of personalized therapy. Specifically the invention is directed to predicting whether an individual having Fragile X Syndrome will clinically respond to treatment with a particular therapeutic agent.

### BACKGROUND OF THE INVENTION

Fragile X syndrome (FXS) is the most common cause of inherited mental retardation with a worldwide prevalence of 1/4000 in males and 1/8000 in females. The incidence of FXS is 10-20 times higher than other X-linked mental retardations. FXS is a monogenetic disease and mainly caused by a CGG-repeat expansion that triggers hypermethylation and silencing of the fragile X mental retardation 1 (FMR1) gene. The absence of the FMR1 protein (FMRP) may result in overstimulation of protein synthesis mediated by metabotropic glutamate receptor 5 (mGluR5) signaling and consequently lead to the diversity of FXS phenotypes. mGluR5 antagonists have the potential to reduce the mGluR5 signaling and normalize the deficits caused by the lack of fragile X mental retardation protein.

There is no specific treatment for FXS and clinical practice varies in different countries. The most frequent medications used to treat the FXS symptoms are stimulants (i.e. methylphenidate), selective serotonin reuptake inhibitors (SSRIs) (e.g. fluoxetine), alpha-adrenoreceptor agonists (e.g., clonidine), mood stabilizers (e.g. carbamazepine), and antipsychotic medication (e.g., risperidone, olazapine). The use of any of these drugs is compromised by their limited efficacy and the potential for undesirable side effects. In recent years, a role for mGluR antagonists has also been suggested.

There is an increasing body of evidence that suggests a patient's genetic profile can be determinative to a patient's responsiveness to a therapeutic treatment. Given the numerous therapies available to an individual having FXS, a determination of the genetic factors that influence, for example, response to a particular drug, could be used to provide a patient with a personalized treatment regime. Such personalized treatment regimes offer the potential to maximize therapeutic benefit to the patient while minimizing related side effects that can be associated with alternative treatment regimes. Thus, there is a need to identify factors which can be used to predict whether a patient is likely to respond to a particular therapy.

### SUMMARY OF THE INVENTION

The present invention is based on the finding that particular biomarkers can be used to select those individuals having FXS who are likely to respond to treatment with an mGluR5 antagonist. Specifically, it was found that the methylation status of the fragile X mental retardation 1 (FMR1) gene region and/or the reduction in the level of FMR1 gene expression and/or the reduction in the amount of FMR1 protein (FMRP) in a sample from an individual having FXS compared to a control, can be used to predict whether that individual will respond to mGluR5 treatment. The present invention thus allows a treatment provider to identify those individuals having FXS who are responders to mGluR5 treatment, and those who are non-responders to such treatment, prior to administration of an mGluR5 antagonist.

In one aspect, the invention includes a method for determining responsiveness of an individual with FXS to treatment with an mGluR5 antagonist. The method includes providing a nucleic acid sample from the individual having Fragile X Syndrome; determining the extent of methylation of a fragile X mental retardation 1 (FMR1) gene region in the sample; and assigning the individual as an mGluR5 responder if all, or predominantly all, of the FMR1 gene region present in the sample is methylated. The extent of methylation of the FMR1 promoter can be determined by any method known in the art including an assay selected from methylation-sensitive restriction enzyme digestion combined with at least one of: Southernblot or quantitative PCR (probe- or SYBR green-based) or from bisulfite DNA modification combined with at least one of: methylation specific PCR (MSP), quantitative methylation specific PCR (probe- or SYBR green-based) or pyrosequencing. In one example, the extent of methylation is determined using a qualitative assay such as MSP and an individual is identified to be an mGluR5 responder if only the FMR1 gene region of interest is detected to be methylated, i.e., no unmethylated FMR1 is detected in the FMR1 gene region of interest. In another example, the extent of methylation is determined using a quantitative assay and an individual is identified to be an mGluR5 responder if the level of methylation of the FMR1 gene region is determined to be 99.5%, or above. An example, of a quantitative assay is a methylation-sensitive restriction enzyme digestion combined with qPCR.

In another aspect, the invention includes a method for determining responsiveness of an individual with FXS to treatment with an mGluR5 antagonist, the method including providing a nucleic acid sample from an individual having FXS; determining the extent of methylation of a fragile X mental retardation 1 (FMR1) gene region in the sample, wherein if all, or predominantly all, of the FMR1 gene region present in the sample is methylated that individual is identified to be an mGluR5 responder; and administering an mGluR5 antagonist to an individual identified to be an mGluR5 responder.

In yet another aspect, the invention includes a method for determining responsiveness of an individual with FXS to treatment with an mGluR5 antagonist, the method including providing a nucleic acid sample from an individual having FXS; and determining the extent of methylation of a fragile X mental retardation 1 (FMR1) gene region in the sample using a methylation sensitive analyzer, wherein if all, or predominantly all, of the FMR1 gene region present in the sample is methylated that individual is identified to be an mGluR5 responder.

In yet another aspect, the invention includes a method for determining responsiveness of an individual with FXS to treatment with an mGluR5 antagonist, the method including providing a nucleic acid sample from an individual having FXS; determining the extent of methylation of a fragile X mental retardation 1 (FMR1) gene region in the sample, wherein if all, or if the level of methylation of the FMR1 gene region is determined to be 99.5%, or above, or have a delta ct of 8 or above, the individual is identified to be an mGluR5 responder and administering an mGluR5 antagonist to an individual identified to be an mGluR5 responder.

In still yet another aspect, the invention includes a method for determining responsiveness of an individual with FXS to treatment with an mGluR5 antagonist, the method including providing a nucleic acid sample from an individual having FXS; determining the extent of methylation of a fragile X mental retardation 1 (FMR1) gene region in the sample, wherein the level of methylation in the sample relative to a control is indicative whether the individual is an mGluR5 responder.

In another aspect, the invention includes a method of determining the responsiveness of an individual with FXS to treatment with an mGluR5 antagonist, the method includes isolating an RNA sample from the individual having Fragile X Syndrome; performing an assay which detects FMR1 mRNA transcripts in the RNA sample, and assigning the individual as an mGluR5 responder if no FMR1 mRNA transcripts are detected or a reduced level of FMR1 mRNA expression is detected as compared to a control. mRNA transcripts can be detected using any method known in the art including Northern blot analysis, reverse transcription-polymerase chain reaction (RT-PCR), RT-PCR ELISA, TaqMan-based quantitative RT-PCR (probe-based quantitative RT-PCR) and SYBR green-based quantitative RT-PCR.

In yet another aspect, the invention includes a method of determining the responsiveness of an individual with FXS to treatment with an mGluR5 antagonist, the method includes isolating a sample from the individual having Fragile X Syndrome; performing an assay which detects FMR1 protein in the sample; and assigning the individual as an mGluR5 responder if the sample lacks the presence of an FMR1 protein (FMRP) or has a reduced amount compared to a control. FMRP detection can be done by any method known in the art including ELISA, flow cytometry, blood smear test (immunostaining), Western blot, HPLC, and mass spectrometry.

In any of the methods described herein the mGluR5 antagonist can be (−)-(3aR,4S,7aR)-4-Hydroxy-4-m-tolylethynyl-octahydro-indole-1-carboxylic acid methyl ester.

### BRIEF DESCRIPTION OF THE DRAWINGS

FIG. 1 depicts the FMR1 promoter and 5′ UTR sequence.

FIG. 2 depicts a bar graph showing the temperature dependent signal kinetics for human FMRP protein detection by F4055-H0002332-M03 antibody combination.

FIG. 3 depicts a bar graph showing the temperature dependent signal kinetics for human FMRP protein detection by MAB2160-F4055 antibody combination.

FIG. 4 depicts a bar graph showing endogenous human FMRP protein detection in primary human fibroblasts.

### DETAILED DESCRIPTION OF THE INVENTION

The present invention is based, in part, on the finding that individuals having Fragile X Syndrome (FXS) whose FMR1 gene is transcriptionally silenced are likely to respond to treatment with an mGluR5 antagonist. Accordingly, the present invention is directed to a method of predicting whether an individual having FXS is an mGluR5 responder. The extent of methylation of the FMR1 gene region, the lack of FMR1 mRNA expression, and the lack of FMR1 protein (FMRP) in a sample of interest can serve individually, or in combination, as biomarkers to predict a patient's responsiveness to an mGluR5 antagonist.

As used herein, “an mGluR5 responder” is an individual having FXS who is likely following therapeutic treatment with an mGluR5 antagonist to show improved behavioral symptoms as assessed using the Aberrant Behavior Checklist—Community Edition (ABC-C) measure of behavior (Bihm et al., Am. J. Ment Retard 96:209-211). The ABC-C measurement looks at various behaviors including stereotypic behavior, hyperactivity, inappropriate speech, and restricted interests. An individual who shows a decrease in ABC-C scores following treatment with an mGluR5 antagonist is classified as an mGluR5 responder. The behavioral symptoms may also be assessed by other methods, such as Clinical Global Impression (CGI) scale, Social Responsiveness Scale (SRS), or Repetitive Behavior Scale-Revised (RBS-R). Individuals showing an improvement according to these tests will also be determined to be an mGluR5 responder.

mGluR5 Antagonists

The present invention can be used to determine which individuals having FXS are likely to respond to treatment with an mGluR5 antagonist. Examples of mGluR5 antagonists include eptidomimetics, proteins, peptides, nucleic acids, small molecules, or other drug candidates. An example of an mGluR5 antagonist is (−)-(3aR,4S,7aR)-4-Hydroxy-4-m-tolylethynyl-octahydro-indole-1-carboxylic acid methyl ester. The mGluR5 antagonist, (−)-(3aR,4S,7aR)-4-Hydroxy-4-m-tolylethynyl-octahydro-indole-1-carboxylic acid methyl ester, as well as methods of making the same, are disclosed in U.S. Pat. No. 7,348,353, which disclosure is incorporated by reference herein. The mGluR5 antagonist, (−)-(3aR,4S,7aR)-4-Hydroxy-4-m-tolylethynyl-octahydro-indole-1-carboxylic acid methyl ester has the following structural formula:

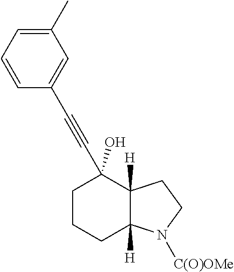

Other mGLUR5 antagonists such as those disclosed in U.S. Pat. No. 7,348,353 are contemplated for use in the methods of the present invention.

In one embodiment, the mGluR5 antagonist is a compound of the formula (I)

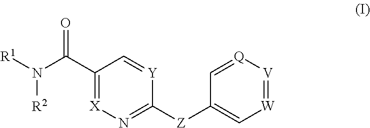

wherein
R¹ represents optionally substituted alkyl or optionally substituted benzyl; and
R² represents hydrogen (H), optionally substituted alkyl or optionally substituted benzyl; or
R¹ and R² form together with the nitrogen atom to which they are attached an optionally substituted heterocycle with less than 14 ring atoms;
R³ represents halogen, alkyl, alkoxy, alkylamino or dialkylamino;
R⁴ represents hydroxy (OH), halogen, alkyl or alkoxy;
Q represents CH, CR⁴ or N;
V represents CH, CR⁴ or N;
W represents CH, CR⁴ or N;
X represents CH or N;
Y represents CH, CR³ or N;
Z represents CH₂, NH or O; and
provided that Q, V and W are not N at the same time;
in free base or acid addition salt form.

In another embodiment, the mGluR5 antagonist is a compound of the formula (II), wherein a compound of the formula (II) is a compound of formula (I) in which at least one of Q, V and W is N; in free base or acid addition salt form.

In yet a further embodiment, mGluR5 antagonist is a compound of the formula (III), wherein the compound of formula (III) is a compound of formula (II) in which Y is CR³; in free base or acid addition salt form.

Preferred substituents, preferred ranges of numerical values or preferred ranges of the radicals present in the formula (I), (II) and (III) and the corresponding intermediate compounds are defined below.

X preferably represents CH.

Y preferably represents CH or CR³, wherein R³ preferably represents halogen, particular preferably chloro.

Z preferably represents NH.

R³ preferably represents fluoro, chloro, C₁₋₄ alkyl, e.g. methyl.

R³ particularly preferably represents chloro.

R¹ and R² preferably form together with the nitrogen atom to which they are attached an unsubstituted or substituted heterocycle having 3-11 ring atoms and 1-4 hetero atoms; the hetero atoms being selected from the group consisting of N, O, S, the substituents being selected from the group consisting of oxo (═O), hydroxy, halogen, amino, nitro, cyano, C₁₋₄ alkyl, C₁₋₄ alkoxy, C₁₋₄ alkoxyalkyl, C₁₋₄ alkoxycarbonyl, C₁₋₄ alkoxycarbonylalkyl, C₁₋₄ halogenalkyl, C₆₋io aryl, halogen-C₆₋io aryl, C₆₋io aryloxy and C₆.io-aryl-C₁₋₄ alkyl.

R¹ and R² form together with the nitrogen atom to which they are attached form an unsubstituted, a single or twofold substituted heterocycle having 5-9 ring atoms and 1-3 hetero atoms; the hetero atoms being selected from the group consisting of N and O; the substituents being selected from the group consisting of halogen and C₁₋₄ alkyl.

R¹ and R² preferably form together with the nitrogen atom to which they are attached an unsubstituted, a single or twofold substituted heterocycle selected from the group consisting of

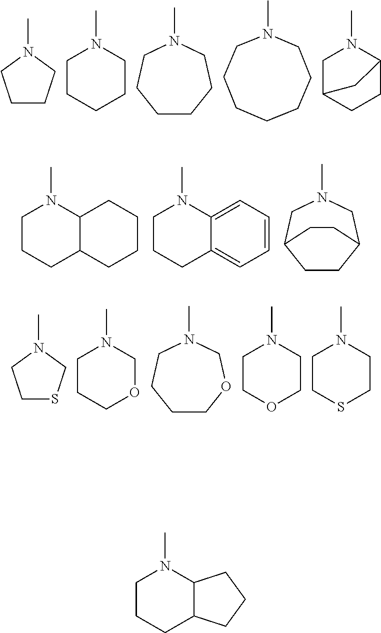

and the substituents being selected from the group consisting of fluoro, chloro, methyl, ethyl, propyl, butyl, trifluoromethyl, fluoropropyl and difluoropropyl.

R¹ and R² preferably represent, independently from each other, C₁-C₄ alkyl or benzyl, optionally substituted by C₁-C₄ BIkOXy or halogen.

The above mentioned general or preferred radical definitions apply both to the end products of the formulae (I), (II) and (III) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formulae (I), (II) and (III) which contain a combination of the meanings mentioned above as being preferred.

Particular preference according to the invention is given to compounds of the formulae (I), (II) and (III) which contain a combination of the meanings listed above as being particularly preferred.

Very particular preference according to the invention is given to the compounds of the formula (I) which contain a combination of the meanings listed above as being very particularly preferred.

Preferred are those compounds of formulae (I), (II) and (III) wherein R² represents an unsubstituted or substituted heterocycle.

Particular preferred are compounds of formulae (IIa to IIe) as shown below:

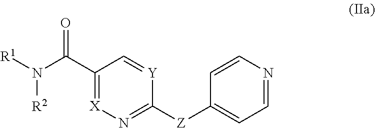

wherein the substituents have the meaning given in this specification;

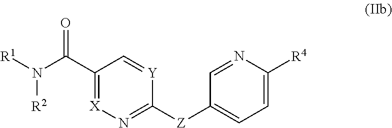

wherein the substituents have the meaning given in this specification;

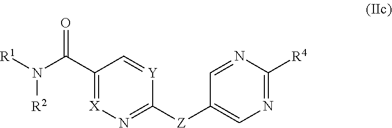

wherein the substituents have the meaning given in this specification;

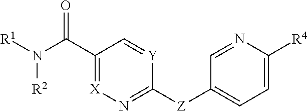

(Hd) wherein R⁴ represents C₁-C₄ alkyl, preferably methyl, and the other substituents have the meaning given in this specification;

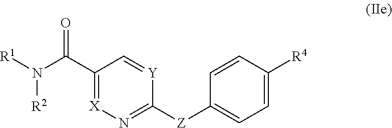

wherein R⁴ represents halogen, preferably chloro, and the other substituents have the meaning given in this specification.

Further preferred compounds of the present invention have the formulae (IIIa to IIIe) as shown below:

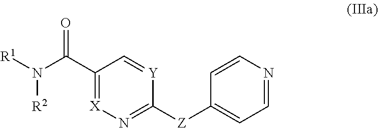

wherein all of the substituents have the meaning given in this specification;

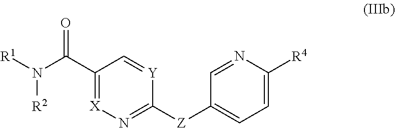

wherein the substituents have the meaning given in this specification;

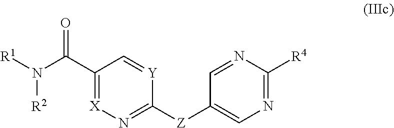

wherein the substituents have the meaning given in this specification;

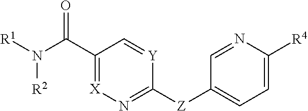

(IIId) wherein R⁴ represents C₁-C₄ alkyl, preferably methyl, and the other substituents have the meaning given in this specification;

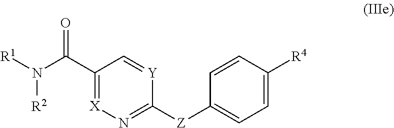

wherein R⁴ represents halogen, preferably chloro, and the other substituents have the meaning given in this specification.

Particular compounds of the formulae (I), (II) and (III) include those described in the Examples given herein.

In another embodiment, the mGluR5 antagonist is a compound of the formula (IV):

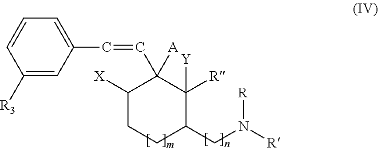

wherein
m is 0 or 1,
n is 0 or 1 and
A is hydroxy
X is hydrogen and
Y is hydrogen, or
A forms a single bond with X or with Y;
R₀ is hydrogen, (C₁₋₄)alkyl, (C₁₋₄)alkoxy, trifluoromethyl, halogen, cyano, nitro, —COOR₁ wherein R₁ is (C₁₋₄)alkyl or —COR₂ wherein R₂ is hydrogen or (C₁₋₄)alkyl, and
R is —COR₃, —COOR₃, —CONR₄R₅ Or —SO₂R₆, wherein R₃ is (C₁₋₄)alkyl, (C₃₋₇)cycloalkyl or optionally substituted phenyl, 2-pyridyl or 2-thienyl; R₄ and R₅, independently, are hydrogen or (C₁₋₄)alkyl; and R₆ is (C₁₋₄)alkyl, (C₃₋₇)cycloalkyl or optionally substituted phenyl, R′ is hydrogen or (Ĉalkyl and
R″ is hydrogen or (C₁₋₄alkyl, or
R′ and R″ together form a group —CH₂—(CH₂)ₘ— wherein m is 0, 1 or 2, in which case one of n and m is different from 0,
with the proviso that R₀ is different from hydrogen, trifluoromethyl and methoxy when n is 0, A is hydroxy, X and Y are both hydrogen, R is COOEt and R′ and R″ together form a group —′(CHz)₂-, in free base or acid addition salt form.

Exemplary compounds of formula (IV) include:
(−)-(3 aR,4S,7aR)-4-Hydroxy-4-m-tolylethynyl-octahydro-indole-1-carboxylic acid methyl ester (−)-(3 aR,4S,7aR)-4-Hydroxy-4-m-tolylethynyl-octahydro-indole-1-carboxylic acid ethyl ester
(−)-(3aR,4S,7aR)-Furan-2-yl-(4-hydroxy-4-m-tolylethynyl-octahydro-indol-1-yl)-methanone
(±)-(3aRS,4SR,7aRS)-4-(3-Chlorophenylethynyl)-4-hydroxy-octahydro-indole-1-carboxylic acid ethyl ester
(±)-(3aRS,4SR,7aRS)-4-(3-Fluoro-phenylethynyl)-4-hydroxy-octahydro-indole-1-carboxylic acid ethyl ester
(SaRŜSRJaRŜ-Hydroxŷ-phenylethynyl-octahydro-indole-1-carboxylic acid(S)(tetrahydrofuran-3-yl)ester
(SaRŜSRJaRŜ-Hydroxŷ-phenylethynyl-octahydro-indole-1-carboxylic acid(R)(tetrahydrofuran-3-yl)ester
(3aRS,4SR,7aRS)-4-Hydroxy-4-(3-chlorophenylethynyl)-octahydro-indol-1-carboxylic acid-(S)(tetrahydrofuran-3yl)ester
(±)-(3aRS,4SR,7aRS)-4-Hydroxy-4-m-tolylethynyl-octahydro-indole-1-carboxylic acid ethyl ester
(±)-(3aRS,4SR,7aRS)-4-(4-Fluoro-phenylethynyl)-4-hydroxy-octahydro-indole-1-carboxylic acid ethyl ester
(±)-(3aRS,4SR,7aRS)-4-(3-chlorophenylethynyl)-4-hydroxy-1-methanesulfonyl-octahydro-indole
(±)-(3aRS,7aRS)-4-Phenylethynyl-2,3,3a,6,7,7a-hexahydro-indole-1-carboxylic acid ethyl ester and
(±)-(RS)-4-phenylethynyl-2,3,5,6,7,7a-hexahydro-indole-1-carboxylic acid ethyl ester (±J-CSRSJaRSĴ̂̂-Trifluoro-1-Ĉphenylethynyl̂.S.Sa.ej.ya-hexahydro-indol-1-yl)-ethanone
(±)-(RS)-4-m-Tolylethynyl-2,3,5,6,7,7a-hexahydro-indole-1-carboxylic acid ethyl ester (±)-(3RS,7aRS)-4-m-Tolylethynyl-2,3,3a,6,7,7a-hexahydro-indole-1-carboxylic acid ethyl ester
(±)-(3RS, 7aRS)-4-(4-Chloro-phenylethynyl)-2,3,3a,6,7,7a-hexahydro-indole-1-carboxylic acid ethyl ester
(±)-(3RS, 7aRS)-4-(2-Fluoro-phenylethynyl)-2,3,3a,6,7,7a-hexahydro-indole-1-carboxylic acid ethyl ester
(±)-(3RS,7aRS)-4-(3-Fluoro-phenylethynyl)-2,3,3a,6,7,7a-hexahydro-indole-1-carboxylic acid ethyl ester
(±)-(RS)-4-(3-Fluoro-phenylethynyl)-2,3,5,6,7,7a-hexahydro-indole-1-carboxylic acid ethyl ester
(±)-(3RS,7aRS)-4-(3-Methoxy-phenylethynyl)-2,3,3a,6,7,7a-hexahydro-indole-1-carboxylic acid ethyl ester
(±)-(RS)-4-(3-Methoxy-phenylethynyl)-2,3,5,6,7,7a-hexahydro-indole-1-carboxylic acid ethyl ester
(±)-(3aRS,4RS,7aSR)-4-Hydroxy-4-phenylethynyl-octahydro-isoindole-2-carboxylic acid ethyl ester
(±)-(3aRS,4RS,7aSR)-4-Hydroxy-4-m-tolylethynyl-octahydro-isoindole-2-carboxylic acid ethyl ester
(±)-(3aRS,4RS,7aSR)-4-Hydroxy-4-p-tolylethynyl-octahydro-isoindole-2-carboxylic acid ethyl ester
(±)-(3aRS,4RS,7aSR)-4-(3-Cyano-phenylethynyl)-4-hydroxy-octahydro-isoindole-2-carboxylic acid ethyl ester
(±HSaRŜRSJaSRM-HydroxŷS-methoxy-phenylethynyO-octahydro-isoindolê-carboxylic acid ethyl ester
(±HSaRŜRSJaSRM-CS-Fluoro-phenylethynyl̂-hydroxy-octahydro-isoindolê-carboxylic acid ethyl ester
(±)-(3aRS,4RS,7aSR)-4-Hydroxy-4-phenylethynyl-octahydro-isoindole-2-carboxylic acid tert-butyl ester
(±)-(3aRS,4RS,7aSR)-4-Hydroxy-4-m-tolylethynyl-octahydro-isoindole-2-carboxylic acid tert-butyl ester (±)-(3aRS,4RS,7aSR)-4-Hydroxy-4-m-tolylethynyl-octahydro-isoindole-2-carboxylic acid methyl ester
(±)-(3aRS,4RS,7aSR)-Furan-2-yl-(4-hydroxy-4-m-tolylethynyl-octahydro-isoindol-2-yl)-methanone
(±ĴSaRŜRS.yaSRJ-Cyclopropyl̂-hydroxŷ-m-tolylethynyl-octahydro-isoindol̂-yl)-methanone
(±)-(3aRS,4RS,7aSR)-(4-Hydroxy-4-m-tolylethynyl-octahydro-isoindol-2-yl)-pyridin-3-yl-methanone
(±)-((1SR,3SR)-3-Hydroxy-3-/r7-tolylethynyl-cyclohexyl)-methyl-carbamic acid methyl ester and
(±)-((1RS,3SR)-3-hydroxy-3-/n-tolylethynyl-cyclohexyl)-methyl-carbamic acid methyl ester
(±)-(1RS,3SR)-((3-Hydroxy-3-/n-tolylethynyl-cyclohexyl)-(4-methoxy-benzyl)-carbamic acid ethyl ester
(±)-(1RS,3RS)-((3-Hydroxy-3-/r7-tolylethynyl-cyclohexyl)-(4-methoxy-benzyl)-carbamic acid ethyl ester
(±)-[(1RS,3SR)-3-Hydroxy-3-(3-methoxy-phenylethynyl)-5,5-dimethyl-cyclohexyl]-methyl-carbamic acid methyl ester
(±)-(1RS,3SR)-(3-Hydroxy-5,5-dimethyl-3-/r7-tolylethynyl-cyclohexyl)-methyl-carbamic acid methyl ester
(±)-[(1RS,3 SR)-3-(3-Fluoro-phenylethynyl)-3-hydroxy-5,5-dimethyl-cyclohexyl]-methyl-carbamic acid methyl ester
(±)-[(1RS,3RS)-3-(3-Fluoro-phenylethynyl)-3-hydroxy-cyclohexyl]-methyl-carbamic acid methyl ester
(±)-[(1RS,3 SR)-3-(3-Fluoro-phenylethynyl)-3-hydroxy-cyclohexyl]-methyl-carbamic acid methyl ester
(±)-[(1RS,3RS)-3-Hydroxy-3-(3-methoxy-phenylethynyl)-cyclohexyl]-methyl-carbamic acid methyl ester
(±)-[(1RS,3 SR)-3-Hydroxy-3-(3-methoxy-phenylethynyl)-cyclohexyl]-methyl-carbamic acid methyl ester
(±)-[(1RS,3RS)-3-(3-Chloro-phenylethynyl)-3-hydroxy-cyclohexyl]-methyl-carbamic acid methyl ester
(±)-[(1RS,3 SR)-3-(3-Chloro-phenylethynyl)-3-hydroxy-cyclohexyl]-methyl-carbamic acid methyl ester (±)-(1RS,3RS)—N-(3-hydroxy-3-m-tolylethynyl-cyclohexyl)-acetamide
(±)-(1RS,3 SR)—N-(3-hydroxy-3-m-tolylethynyl-cyclohexyl)-acetamide
(±)-(1RS,3 RS)-(3-Hydroxy-3-m-tolylethynyl-cyclohexyl)-carbamic acid ethyl ester
(±)-(1RS,3 SR)-(3-Hydroxy-3-m-tolylethynyl-cyclohexyl)-carbamic acid ethyl ester
(±)-(1RS,3RS)-[3-(3-Fluoro-phenylethynyl)-3-hydroxy-cyclohexyl]-carbamic acid ethyl ester
(±)-(1RS,3SR)-[3-(3-Fluoro-phenylethynyl)-3-hydroxy-cyclohexyl]-carbamic acid ethyl ester
(±)-(1RS,3RS)-[3-(3-Methoxy-phenylethynyl)-3-hydroxy-cyclohexyl]-carbamic acid ethyl ester
(±)-(1RS,3RS)—N-[3-(3-Fluoro-phenylethynyl)-3-hydroxy-cyclohexyl]-acetamide.
(±)-(1RS,3 SR)—N-[3-(3-Fluoro-phenylethynyl)-3-hydroxy-cyclohexyl]-acetamide
(±)-(1RS,3SR)-[3-Hydroxy-3-(3-methoxy-phenylethynyl)-cyclohexyl]-carbamic acid ethyl ester
(±)-(1RS,3RS)—N-[3-Hydroxy-3-(3-methoxy-phenylethynyl)-cyclohexyl]-acetamide
(±)-(1RS,3SR)—N-[3-Hydroxy-3-(3-methoxy-phenylethynyl)-cyclohexyl]-acetamide.
(±)-(1RS,3RS)-[3-Hydroxy-3-(3-methoxy-phenylethynyl)-cyclohexyl]-carbamic acid tert-butyl ester
(±)-(1RS,3SR)-[3-Hydroxy-3-(3-methoxy-phenylethynyl)-cyclohexyl]-carbamic acid tert-butyl ester
(±)-(1RS,3RS)-(3-Hydroxy-3-m-tolylethynyl-cyclohexyl)-carbamic acid tert-butyl ester (±)-(1 RS,3SR)-(3-Hydroxy-3-m-tolylethynyl-cyclohexyl)-carbamic acid tert-butyl ester (±)-(1RS,3RS)-(3-(3-Fluoro-phenylethynyl)-3-hydroxy-cyclohexyl]-carbamic acid tert-butyl ester
(±)-(1RS,3SR)-(3-(3-Fluoro-phenylethynyl)-3-hydroxy-cyclohexyl]-carbamic acid tert-butyl ester
(±)-(1RS,3RS)-[3-(3-Fluoro-phenylethynyl)-3-hydroxy-cyclohexyl]-carbamic acid methyl ester (±)-(1RS,3SR)-[3-(3-Fluoro-phenylethynyl)-3-hydroxy-cyclohexyl]-carbamic acid methyl ester (±)-(3-Phenylethynyl-cyclohex-2-enyl)-carbamic acid ethyl ester and (±)-3-phenylethynyl-cyclohex-3-enyl)-carbamic acid ethyl ester
(±)-Methyl-(3-phenylethynyl-cyclohex-3-enyl)-carbamic acid ethyl ester
(±)-(4aRS,5RS,8aSR)-5-Hydroxy-5-phenylethynyl-octahydro-quinoline-1-carboxylic acid ethyl ester
(±)-[(4aRS,5SR,8aSR)-5-(3-Chloro-phenylethynyl)-5-hydroxy-octahydro-quinolin-1-yl]-furan-2-yl-methanone (±)-[(4aRS,5RS,8aSR)-5-(3-Chloro-phenylethynyl)-5-hydroxy-octahydro-quinolin-1-yl]-furan-2-yl-methanone
(±)-(4aRS,5RS,8aSR)-5-(3-Chloro-phenylethynyl)-5-hydroxy-octahydro-quinoline-1-carboxylic acid tert-butyl ester
(±)-[(4aRS,5SR,8aSR)-5-(3-Chloro-phenylethynyl)-5-hydroxy-octahydro-quinolin-1-yl]-morpholin-4-yl-methanone
(±)-[(4aRS,5SR,8aSR)-5-(3-chloro-phenylethynyl)-5-hydroxy-octahydro-quinolin-1-yl]-(4-methyl-piperazin-1-yl)-methanone
(±)-(4aRS,5RS,8aSR)-5-(3-chloro-phenylethynyl)-5-hydroxy-octahydro-quinoline-1-carboxylic acid ethyl ester and (±)-(4aRS,5SR,8aSR)-5-(3-chloro-phenylethynyl)-5-hydroxy-octahydro-quinoline-1-carboxylic acid ethyl ester
(±)-(4aRS,5SR,8aSR)-5-Hydroxy-5-m-tolylethynyl-octahydro-quinoline-1-carboxylic acid ethyl ester
(±)-(4aRS,5RS,8aSR)-5-Hydroxy-5-m-tolylethynyl-octahydro-quinoline-1-carboxylic acid ethyl ester.

In a further embodiment, the mGluR modulator is a compound of the formula (V):

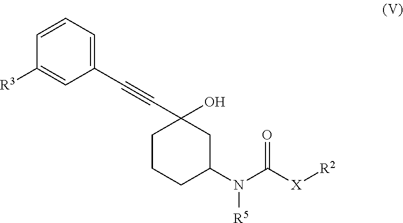

wherein
R¹ represents hydrogen or alkyl;
R² represents an unsubstituted or substituted heterocycle or
R² represents an unsubstituted or substituted aryl;
R³ represents alkyl or halogen;
X represents a single bond or an alkandiyl-group, optionally interrupted by one or more oxygen atoms or carbonyl groups or carbonyloxy groups in free base or acid addition salt form.

Exemplary compounds of formula (V) include:
Furan-3-carboxylic acid [(1S,3S)-3-(3-chloro-phenylethynyl)-3-hydroxy-cyclohexyl]-amide Furan-2-carboxylic acid [(1R,3R)-3-(3-chloro-phenylethynyl)-3-hydroxy-cyclohexyl]-amide Furan-2-carboxylic acid [(1S,3S)-3-(3-chloro-phenylethynyl)-3-hydroxy-cyclohexyl]-amide 3H-Imidazole-4-carboxylic acid [(1R,3R)-3-(3-chloro-phenylethynyl)-3-hydroxy-cyclohexyl]-amide
3H-Imidazole-4-carboxylic acid [(1S,3S)-3-(3-chloro-phenylethynyl)-3-hydroxy-cyclohexyl]-amide
4H-[1,2,4]Triazole-3-carboxylic acid [(1R,3R)-3-(3-chloro-phenylethynyl)-3-hydroxy-cyclohexyl]-amide
4H-[1,2,4]Triazole-3-carboxylic acid [(1S,3S)-3-(3-chloro-phenylethynyl)-3-hydroxy-cyclohexyl]-amide
2-Methyl-furan-3-carboxylic acid [(±)-(1R,3R)-3-(3-chloro-phenylethynyl)-3-hydroxy-cyclohexyl]-amide
N-[(±)-(1R,3R)-3-(3-Chloro-phenylethynyl)-3-hydroxy-cyclohexyl]-3,4-difluoro-benzamide Benzo[1,3]dioxole-2-carboxylic acid [(±)-(1R,3R)-3-(3-chloro-phenylethynyl)-3-hydroxy-cyclohexyl]-amide
5-Methyl-pyrazine-2-carboxylic acid [(±)-(1R,3R)-3-(3-chloro-phenylethynyl)-3-hydroxy-cyclohexyl]-amide
Quinoxaline-2-carboxylic acid [(±)-(1R,3R)-3-(3-chloro-phenylethynyl)-3-hydroxy-cyclohexyl]-amide
Benzofuran-2-carboxylic acid [(±)-(1R,3R)-3-(3-chloro-phenylethynyl)-3-hydroxy-cyclohexyl]-amide
Benzooxazole-2-carboxylic acid [(±)-(1R,3R)-3-(3-chloro-phenylethynyl)-3-hydroxy-cyclohexyl]-amide
2,5-Dimethyl-furan-3-carboxylic acid [(±)-(1R,3R)-3-(3-chloro-phenylethynyl)-3-hydroxy-cyclohexyl]-amide
(R,S)-Tetrahydro-furan-3-carboxylic acid [(±)-(1R,3R)-3-(3-chloro-phenylethynyl)-3-hydroxy-cyclohexyl]-amide
Furan-3-carboxylic acid ((1R.SRJ-S-hydroxy-S-m-tolylethynyl-cyclohexyO-amide
Furan-3-carboxylic acid ((1S,3S)-3-hydroxy-3-m-tolylethynyl-cyclohexyl)-amide Furan-3-carboxylic acid ((±)-(1R.SRJ-S-hydroxy-S-m-tolylethynyl-cyclohexyO-amide
Furan-2-carboxylic acid ((1R,3R)-3-hydroxy-3-m-tolylethynyl-cyclohexyl)-amide
Furan-2-carboxylic acid ((1S,3S)-3-hydroxy-3-m-tolylethynyl-cyclohexyl)-amide
Furan-2-carboxylic acid ((±)-(1R,3R)-3-hydroxy-3-m-tolylethynyl-cyclohexyl)-amide Isoxazole-5-carboxylic acid ((1R,3R)-3-hydroxy-3-m-tolylethynyl-cyclohexyl)-amide Isoxazole-5-carboxylic acid ((1S,3S)-3-hydroxy-3-m-tolylethynyl-cyclohexyl)-amide Isoxazole-5-carboxylic acid ((±)-(1 R,3R)-3-hydroxy-3-m-tolylethynyl-cyclohexyl)-amide
5-Methyl-pyrazine-2-carboxylic acid ((±)-(1R,3R)-3-hydroxy-3-m-tolylethynyl-cyclohexyl)-amide 4H-[1,2,4]Triazole-3-carboxylic acid ((±)-(1R,3R)-3-hydroxy-3-m-tolylethynyl-cyclohexyl)-amide 3H-Imidazole-4-carboxylic acid ((±)-(1R,3R)-3-hydroxy-3-m-tolylethynyl-cyclohexyl)-amide
Tetrahydro-pyran-4-carboxylic acid ((±)-(1R,3R)-3-hydroxy-3-m-tolylethynyl-cyclohexyl)-amide
1-Methyl-1H-imidazole-4-carboxylic acid ((±)-(1R,3R)-3-hydroxy-3-m-tolylethynyl-cyclohexyl)-amide
(R,S)-Tetrahydro-furan-2-carboxylic acid ((±)-(1R,3R)-3-hydroxy-3-m-tolylethynyl-cyclohexyl)-amide
(R,S)-Tetrahydro-furan-3-carboxylic acid ((±)-(1R,3R)-3-hydroxy-3-m-tolylethynyl-cyclohexyl)-amide
Furan-3-carboxylic acid [(1R,3R)-3-(3-fluoro-phenylethynyl)-3-hydroxy-cyclohexyl]-amide Furan-3-carboxylic acid [(1S,3S)-3-(3-fluoro-phenylethynyl)-3-hydroxy-cyclohexyl]-amide Furan-2-carboxylic acid [(1R,3R)-3-(3-fluoro-phenylethynyl)-3-hydroxy-cyclohexyl]-amide Furan-2-carboxylic acid [(1S,3S)-3-(3-fluoro-phenylethynyl)-3-hydroxy-cyclohexyl]-amide 3H-Imidazole-4-carboxylic acid [(±)-(1R,3R)-3-(3-fluoro-phenylethynyl)-3-hydroxy-cyclohexyl]-amide
N-[(1S,3S)-3-(3-Chloro-phenylethynyl)-3-hydroxy-cyclohexyl]-3,4-difluoro-benzamide N-[(1 R,3R)-3-(3-Chloro-phenylethynyl)-3-hydroxy-cyclohexyl]-3,4-difluoro-benzamide Pyridine-2-carboxylic acid [(1S,3S)-3-(3-chloro-phenylethynyl)-3-hydroxy-cyclohexyl]-amide Pyridine-2-carboxylic acid [(1R,3R)-3-(3-chloro-phenylethynyl)-3-hydroxy-cyclohexyl]-amide N-[(1S,3S)-3-(3-Chloro-phenylethynyl)-3-hydroxy-cyclohexyl]-nicotinamide
N-[(1R,3R)-3-(3-Chloro-phenylethynyl)-3-hydroxy-cyclohexyl]-nicotinamide Benzo[1,3]dioxole-2-carboxylic acid [(1S,3S)-3-(3-chloro-phenylethynyl)-3-hydroxy-cyclohexyl]-amide
5-Methyl-pyrazine-2-carboxylic acid [(1S,3S)-3-(3-chloro-phenylethynyl)-3-hydroxy-cyclohexyl]-amide
2-Methyl-furan-3-carboxylic acid [(1S,3S)-3-(3-chloro-phenylethynyl)-3-hydroxy-cyclohexyl]-amide
(R)-Tetrahydro-furan-2-carboxylic acid [(1R,3R)-3-(3-chloro-phenylethynyl)-3-hydroxy-cyclohexyl]-amide
(S)-Tetrahydro-furan-2-carboxylic acid [(1R,3R)-3-(3-chloro-phenylethynyl)-3-hydroxy-cyclohexyl]-amide
Isoxazole-5-carboxylic acid [(1S,3S)-3-(3-chloro-phenylethynyl)-3-hydroxy-cyclohexyl]-amide 5-Methyl-pyrazine-2-carboxylic acid [(1R,3R)-3-(3-chloro-phenylethynyl)-3-hydroxy-cyclohexyl]-amide
2-Methyl-furan-3-carboxylic acid [(1R,3R)-3-(3-chloro-phenylethynyl)-3-hydroxy-cyclohexyl]-amide
Isoxazole-5-carboxylic acid [(1R,3R)-3-(3-chloro-phenylethynyl)-3-hydroxy-cyclohexyl]-amide 5-Chloro-furan-2-carboxylic acid [(1R,3R)-3-(3-chloro-phenylethynyl)-3-hydroxy-cyclohexyl]-amide 5-Chloro-furan-2-carboxylic acid [(1S,3S)-3-(3-chloro-phenylethynyl)-3-hydroxy-cyclohexyl]-amide
(S)-Tetrahydro-furan-3-carboxylic acid [(1R,3R)-3-(3-chloro-phenylethynyl)-3-hydroxy-cyclohexyl]-amide
(R)-Tetrahydro-furan-3-carboxylic acid [(1R,3R)-3-(3-chloro-phenylethynyl)-3-hydroxy-cyclohexyl]-amide
N-[(1S,3S)-3-(3-Chloro-phenylethynyl)-3-hydroxy-cyclohexyl]-isonicotinamide
N-[(1R,3R)-3-(3-Chloro-phenylethynyl)-3-hydroxy-cyclohexyl]-isonicotinamide
3,5-Difluoro-pyridine-2-carboxylicacid [(1R,3R)-3-(3-chloro-phenylethynyl)-3-hydroxy-cyclohexyl]-amide
3,5-Difluoro-pyridine-2-carboxylicacid [(1S,3S)-3-(3-chloro-phenylethynyl)-3-hydroxy-cyclohexyl]-amide
6-Methyl-pyridine-2-carboxylic acid [(1S,3S)-3-(3-chloro-phenylethynyl)-3-hydroxy-cyclohexyl]-amide 6-Methyl-pyridine-2-carboxylic acid [(1R,3R)-3-(3-chloro-phenylethynyl)-3-hydroxy-cyclohexyl]-amide
S-Chloro-pyridinê-carboxylic acid [(1R,3R)-3-(3-chloro-phenylethynyl)-3-hydroxy-cyclohexyl]-amide
δ-Chloro-pyridinê-carboxylic acid [(1S,3S)-3-(3-chloro-phenylethynyl)-3-hydroxy-cyclohexyl]-amide
δ-Chloro-pyridinê-carboxylic acid [(1R,3R)-3-(3-chloro-phenylethynyl)-3-hydroxy-cyclohexyl]-amide
6-Chloro-pyridine-2-carboxylic acid [(1S,3S)-3-(3-chloro-phenylethynyl)-3-hydroxy-cyclohexyl]-amide
5-Chloro-1-methyl-1H-pyrrole-2-carboxylic acid [(1R,3R)-3-(3-chloro-phenylethynyl)-3-hydroxy-cyclohexyl]-amide
5-Chloro-1-methyl-1H-pyrrole-2-carboxylic acid [(1S,3S)-3-(3-chloro-phenylethynyl)-3-hydroxy-cyclohexyl]-amide
5-Chloro-1H-pyrrole-2-carboxylic acid [(1R,3R)-3-(3-chloro-phenylethynyl)-3-hydroxy-cyclohexyl]-amide
5-Chloro-1H-pyrrole-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-4-dimethyl amino-benzamide
1H-Pyrrole-3-carboxylic acid [(1S,3S)-3-(3-chloro-phenylethynyl)-3-hydroxy-cyclohexyl]-amide
N-[(1S,3S)-3-(3-chloro-phenylethynyl)-3-hydroxy-cyclohexyl]-4-methyl-benzamide
N-[(1S,3S)-3-(3-Chloro-phenylethynyl)-3-hydroxy-cyclohexyl]-4-methyl-benzamide
N-[(1S,3S)-3-(3-Chloro-phenylethynyl)-3-hydroxy-cyclohexyl]-3-fluoro-benzamide
N-[(1S,3S)-3-(3-Chloro-phenylethynyl)-3-hydroxy-cyclohexyl]-2-ethyl-butyramide
N-[(1S,3S)-3-(3-Chloro-phenylethynyl)-3-hydroxy-cyclohexyl]-4-(2,5-dimethoxy-phenyl)-4-oxo-butyramide
2-(2-Benzyloxy-ethoxy)-N-[(1S,3S)-3-(3-chloro-phenylethynyl)-3-hydroxy-cyclohexyl]-acetamide
N-[(1S,3S)-3-(3-Chloro-phenylethynyl)-3-hydroxy-cyclohexyl]-2-phenyl-acetamide
N-[(1S,3S)-3-(3-Chloro-phenylethynyl)-3-hydroxy-cyclohexyl]-3-(1H-indol-4-yl)-propionamide 2-Benzo[1,3]dioxol-5-yl-N-[(1S,3S)-3-(3-chloro-phenylethynyl)-3-hydroxy-cyclohexyl]-acetamide
N-[(1S,3S)-3-(3-Chloro-phenylethynyl)-3-hydroxy-cyclohexyl]-2-phenoxy-propionamide
N-[(1S,3S)-3-(3-Chloro-phenylethynyl)-3-hydroxy-cyclohexyl]-2-(2-fluoro-phenyl)-acetamide
5-Hydroxy-1H-indole-2-carboxylic acid [(1S,3S)-3-(3-chloro-phenylethynyl)-3-hydroxy-cyclohexyl]-amide
1-Methyl-1H-pyrrole-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-terephthalamic acid methyl ester
N-[(1S,3S)-3-(3-Chloro-phenylethynyl)-3-hydroxy-cyclohexyl]-2-(2-trifluoromethoxy-phenyl)-acetamide
5-Chloro-N-[(1S,3S)-3-(3-chloro-phenylethynyl)-3-hydroxy-cyclohexyl]-2-hydroxy-benzamide
N-[(1S,3S)-3-(3-Chloro-phenylethynyl)-3-hydroxy-cyclohexyl]-4-hydroxy-benzamide
N-[(1S,3S)-3-(3-Chloro-phenylethynyl)-3-hydroxy-cyclohexyl]-2-hydroxy-benzamide
4-Amino-N-[(1S,3S)-3-(3-chloro-phenylethynyl)-3-hydroxy-cyclohexyl]-benzamide
4-Amino-5-chloro-N-[(1S,3S)-3-(3-chloro-phenylethynyl)-3-hydroxy-3-Amino-4-chloro-N-[(1S,3S)-3-(3-chloro-phenylethynyl)-3-hydroxy-cyclohexyl]-benzamide
3-Amino-N-[(1S,3S)-3-(3-chloro-phenylethynyl)-3-hydroxy-cyclohexyl]-4-methyl-benzamide
2-Amino-N-[(1S,3S)-3-(3-chloro-phenylethynyl)-3-hydroxy-cyclohexyl]-nicotinamide
N-[(1S,3S)-3-(3-Chloro-phenylethynyl)-3-hydroxy-cyclohexyl]-4-hydroxy-3-methoxy-benzamide
N-[(1S,3S)-3-(3-Chloro-phenylethynyl)-3-hydroxy-cyclohexyl]-2-fluoro-benzamide
N-[(1S,3S)-3-(3-Chloro-phenylethynyl)-3-hydroxy-cyclohexyl]-4-methanesulfonyl-benzamide
Pyridine-2-carboxylic acid [(1S,3S)-3-(3-chloro-phenylethynyl)-3-hydroxy-cyclohexyl]-amide
3-Amino-pyrazine-2-carboxylic acid [(1S,3S)-3-(3-chloro-phenylethynyl)-3-hydroxy-cyclohexyl]-amide
6-Amino-N-[(1S,3S)-3-(3-chloro-phenylethynyl)-3-hydroxy-cyclohexyl]-nicotinamide
4-(4-Amino-benzoylamino)-benzoic acid [(1R,3R)-3-(3-chloro-phenylethynyl)-3-hydroxy-cyclohexyl]-amide
2,6-Dioxo-1,2,3,6-tetrahydro-pyrimidine-4-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-isonicotinamide 3-Chloro-N-[(1S,3S)-3-(3-chloro-phenylethynyl)-3-hydroxy-cyclohexyl]-benzamide
N-[(1S,3S)-3-(3-Chloro-phenylethynyl)-3-hydroxy-cyclohexyl]-2,3-dimethoxy-benzamide
N-[(1S,3S)-3-(3-Chloro-phenylethynyl)-3-hydroxy-cyclohexyl]-4-oxo-4-phenyl-butyramide
2-Chloro-N-[(1S,3S)-3-(3-chloro-phenylethynyl)-3-hydroxy-cyclohexyl]-nicotinamide
5-Bromo-N-[(1S,3S)-3-(3-chloro-phenylethynyl)-3-hydroxy-cyclohexyl]-nicotinamide Isoquinoline-1-carboxylic acid [(1S,3S)-3-(3-chloro-phenylethynyl)-3-hydroxy-cyclohexyl]-amide
Pyrazine-2-carboxylic acid [(1S,3S)-3-(3-chloro-phenylethynyl)-3-hydroxy-cyclohexyl]-amide
3-Benzoyl-pyridine-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-methyl-nicotinamide
Quinoxaline-2-carboxylic acid [(1S,3S)-3-(3-chloro-phenylethynyl)-3-hydroxy-cyclohexyl]-amide
Pyridazine-4-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-methylsulfanyl-nicotinamide
N-[(1S,3S)-3-(3-Chloro-phenylethynyl)-3-hydroxy-cyclohexyl]-4-trifluoromethyl-nicotinamide
2-Chloro-N-[(1S,3S)-3-(3-chloro-phenylethynyl)-3-hydroxy-cyclohexyl]-isonicotinamide
2-Chloro-N-[(1S,3S)-3-(3-chloro-phenylethynyl)-3-hydroxy-cyclohexyl]-6-methyl-nicotinamide
6-Chloro-N-[(1S,3S)-3-(3-chloro-phenylethynyl)-3-hydroxy-cyclohexyl]-nicotinamide
2-Chloro-N-[(1S,3S)-3-(3-chloro-phenylethynyl)-3-hydroxy-cyclohexyl]-6-methyl-isonicotinamide
N-[(1S,3S)-3-(3-Chloro-phenylethynyl)-3-hydroxy-cyclohexyl]-2-(4,5-dimethoxy-3-oxo-1,3-dihydro-isobenzofuran-1-yl)-acetamide
1̂.δ.β-Tetrahydro-cyclopentapyrazole-5-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-3-(1H-indol-2-yl)-propionamide
6-[(1S,3S)-3-(3-Chloro-phenylethynyl)-3-hydroxy-cyclohexylcarbamoyl]-pyridine-2-carboxylic acid isopropyl ester
Quinoline-6-carboxylic acid [(1S,3S)-3-(3-chloro-phenylethynyl)-3-hydroxy-cyclohexyl]-amide
5-Methyl-isoxazole-4-carboxylic acid [(1S,3S)-3-(3-chloro-phenylethynyl)-3-hydroxy-cyclohexyl]-amide Benzofuran-3-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-(2-methoxy-phenoxy)-acetamide.

In a further embodiment, the mGluR modulator is a compound of the formula (VI)

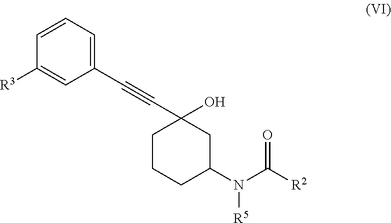

wherein
R¹ represents hydrogen or alkyl;
R R² represents an unsubstituted or substituted heterocycle or
R² represents an unsubstituted or substituted aryl;
R³ represents alkyl or halogen;
in free base or acid addition salt form.

Further examples mGluR5 antagonists include compounds of the formula (I) as defined in WO 2004/014881 and compounds of the formula (I) as defined in WO 2007/021575; the contents of these publications are incorporated herein by reference.

The present invention can be used to determine which individuals having FXS are likely to respond to treatment with an mGluR5 antagonist. Examples of mGluR5 antagonists include eptidomimetics, proteins, peptides, nucleic acids, small molecules, or other drug candidates. An example of an mGluR5 antagonist is (−)-(3aR,4S,7aR)-4-Hydroxy-4-m-tolylethynyl-octahydro-indole-1-carboxylic acid methyl ester. The mGluR5 antagonist, (−)-(3aR,4S,7aR)-4-Hydroxy-4-m-tolylethynyl-octahydro-indole-1-carboxylic acid methyl ester, as well as methods of making the same, are disclosed in U.S. Pat. No. 7,348,353, which disclosure is incorporated by reference herein. The mGluR5 antagonist, (−)-(3aR,4S,7aR)-4-Hydroxy-4-m-tolylethynyl-octahydro-indole-1-carboxylic acid methyl ester has the following structural formula:

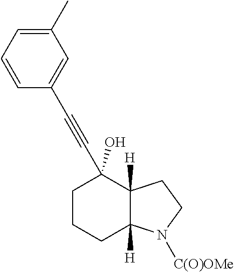

Other mGLUR5 antagonists such as those disclosed in U.S. Pat. No. 7,348,353 are contemplated for use in the methods of the present invention.

In one embodiment, the mGluR5 antagonist is a compound of the formula (I)

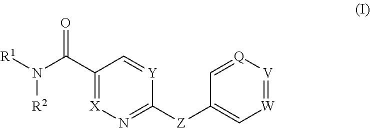

wherein
R¹ represents optionally substituted alkyl or optionally substituted benzyl; and
R² represents hydrogen (H), optionally substituted alkyl or optionally substituted benzyl; or
R¹ and R² form together with the nitrogen atom to which they are attached an optionally substituted heterocycle with less than 14 ring atoms;
R³ represents halogen, alkyl, alkoxy, alkylamino or dialkylamino;
R⁴ represents hydroxy (OH), halogen, alkyl or alkoxy;
Q represents CH, CR⁴ or N;
V represents CH, CR⁴ or N;
W represents CH, CR⁴ or N;
X represents CH or N;
Y represents CH, CR³ or N;
Z represents CH₂, NH or O; and
provided that Q, V and W are not N at the same time;
in free base or acid addition salt form.

In another embodiment, the mGluR5 antagonist is a compound of the formula (II), wherein a compound of the formula (II) is a compound of formula (I) in which at least one of Q, V and W is N; in free base or acid addition salt form.

In yet a further embodiment, mGluR5 antagonist is a compound of the formula (III), wherein the compound of formula (III) is a compound of formula (II) in which Y is CR³; in free base or acid addition salt form.

Preferred substituents, preferred ranges of numerical values or preferred ranges of the radicals present in the formula (I), (II) and (III) and the corresponding intermediate compounds are defined below.

X preferably represents CH.

Y preferably represents CH or CR³, wherein R³ preferably represents halogen, particular preferably chloro.

Z preferably represents NH.

R³ preferably represents fluoro, chloro, C₁₋₄ alkyl, e.g. methyl.

R³ particularly preferably represents chloro.

R¹ and R² preferably form together with the nitrogen atom to which they are attached an unsubstituted or substituted heterocycle having 3-11 ring atoms and 1-4 hetero atoms; the hetero atoms being selected from the group consisting of N, O, S, the substituents being selected from the group consisting of oxo (═O), hydroxy, halogen, amino, nitro, cyano, C₁₋₄ alkyl, C₁₋₄ alkoxy, C₁₋₄ alkoxyalkyl, C₁₋₄ alkoxycarbonyl, C₁₋₄ alkoxycarbonylalkyl, C₁₋₄ halogenalkyl, C₆₋io aryl, halogen-C₆₋io aryl, C₆₋io aryloxy and C₆.io-aryl-C₁₋₄ alkyl.

R¹ and R² form together with the nitrogen atom to which they are attached form an unsubstituted, a single or twofold substituted heterocycle having 5-9 ring atoms and 1-3 hetero atoms; the hetero atoms being selected from the group consisting of N and O; the substituents being selected from the group consisting of halogen and C₁₋₄ alkyl.

R¹ and R² preferably form together with the nitrogen atom to which they are attached an unsubstituted, a single or twofold substituted heterocycle selected from the group consisting of

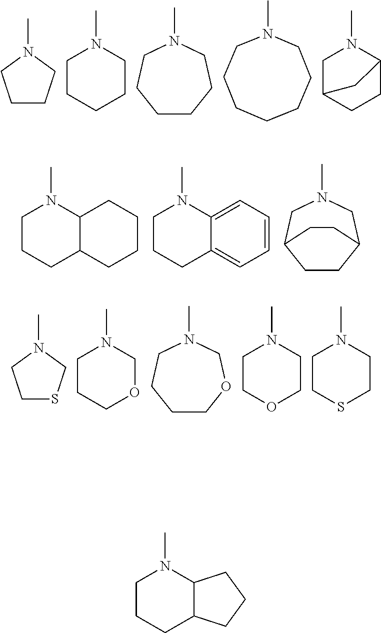

and the substituents being selected from the group consisting of fluoro, chloro, methyl, ethyl, propyl, butyl, trifluoromethyl, fluoropropyl and difluoropropyl.

R¹ and R² preferably represent, independently from each other, C₁-C₄ alkyl or benzyl, optionally substituted by C₁-C₄ BIkOXy or halogen.

The above mentioned general or preferred radical definitions apply both to the end products of the formulae (I), (II) and (III) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formulae (I), (II) and (III) which contain a combination of the meanings mentioned above as being preferred.

Particular preference according to the invention is given to compounds of the formulae (I), (II) and (III) which contain a combination of the meanings listed above as being particularly preferred.

Very particular preference according to the invention is given to the compounds of the formula (I) which contain a combination of the meanings listed above as being very particularly preferred.

Preferred are those compounds of formulae (I), (II) and (III) wherein R² represents an unsubstituted or substituted heterocycle.

Particular preferred are compounds of formulae (IIa to IIe) as shown below:

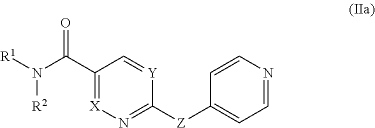

wherein the substituents have the meaning given in this specification;

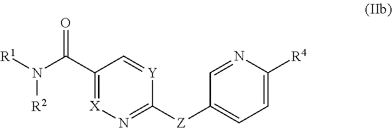

wherein the substituents have the meaning given in this specification;

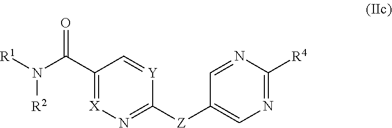

wherein the substituents have the meaning given in this specification;

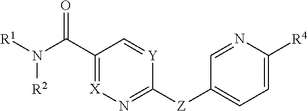

(Hd) wherein R⁴ represents Ci-C₄ alkyl, preferably methyl, and the other substituents have the meaning given in this specification;

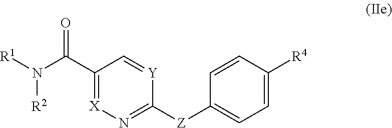

wherein R⁴ represents halogen, preferably chloro, and the other substituents have the meaning given in this specification.

Further preferred compounds of the present invention have the formulae (IIIa to IIIe) as shown below:

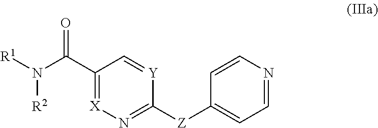

wherein all of the substituents have the meaning given in this specification;

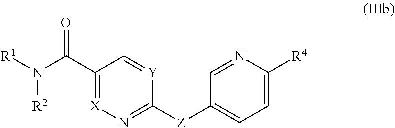

wherein the substituents have the meaning given in this specification;

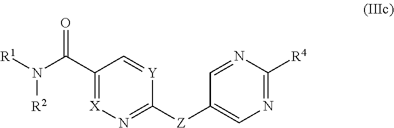

wherein the substituents have the meaning given in this specification;

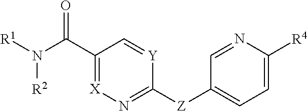

(IIId) wherein R⁴ represents C₁-C₄ alkyl, preferably methyl, and the other substituents have the meaning given in this specification;

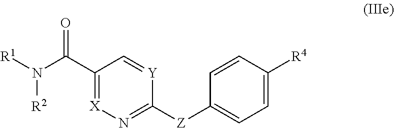

wherein R⁴ represents halogen, preferably chloro, and the other substituents have the meaning given in this specification.

Particular compounds of the formulae (I), (II) and (III) include those described in the Examples given herein.

In another embodiment, the mGluR5 antagonist is a compound of the formula (IV):

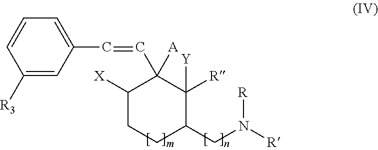

wherein
m is 0 or 1,
n is 0 or 1 and
A is hydroxy
X is hydrogen and
Y is hydrogen, or
A forms a single bond with X or with Y;
R₀ is hydrogen, (C₁₋₄)alkyl, (C₁₋₄alkoxy, trifluoromethyl, halogen, cyano, nitro, —COOR₁ wherein R₁ is (C₁₋₄)alkyl or —COR₂ wherein R₂ is hydrogen or (C₁₋₄)alkyl, and
R is —COR₃, —COOR₃, —CONR₄R₅ Or —SO₂R₆, wherein R₃ is (C₁₋₄)alkyl, (C₃₋₇)cycloalkyl or optionally substituted phenyl, 2-pyridyl or 2-thienyl; R₄ and R₅, independently, are hydrogen or (C₁₋₄)alkyl; and R₆ is (C₁₋₄)alkyl, (C₃₋₇)cycloalkyl or optionally substituted phenyl, R′ is hydrogen or (Ĉalkyl and
R″ is hydrogen or (C₁₋₄alkyl, or
R′ and R″ together form a group —CH₂—(CH₂)ₘ— wherein m is 0, 1 or 2, in which case one of n and m is different from 0,
with the proviso that R₀ is different from hydrogen, trifluoromethyl and methoxy when n is 0, A is hydroxy, X and Y are both hydrogen, R is COOEt and R′ and R″ together form a group —′(CHz)₂-, in free base or acid addition salt form.

Exemplary compounds of formula (IV) include:
(−)-(3 aR,4S,7aR)-4-Hydroxy-4-m-tolylethynyl-octahydro-indole-1-carboxylic acid methyl ester (−)-(3 aR,4S,7aR)-4-Hydroxy-4-m-tolylethynyl-octahydro-indole-1-carboxylic acid ethyl ester
(−)-(3aR,4S,7aR)-Furan-2-yl-(4-hydroxy-4-m-tolylethynyl-octahydro-indol-1-yl)-methanone
(±)-(3aRS,4SR,7aRS)-4-(3-Chlorophenylethynyl)-4-hydroxy-octahydro-indole-1-carboxylic acid ethyl ester
(±)-(3aRS,4SR,7aRS)-4-(3-Fluoro-phenylethynyl)-4-hydroxy-octahydro-indole-1-carboxylic acid ethyl ester
(SaRŜSRJaRŜ-Hydroxŷ-phenylethynyl-octahydro-indole-1-carboxylic acid(S)(tetrahydrofuran-3-yl)ester
(SaRŜSRJaRŜ-Hydroxŷ-phenylethynyl-octahydro-indole-1-carboxylic acid(R)(tetrahydrofuran-3-yl)ester
(3aRS,4SR,7aRS)-4-Hydroxy-4-(3-chlorophenylethynyl)-octahydro-indol-1-carboxylic acid-(S)(tetrahydrofuran-3yl)ester
(±)-(3aRS,4SR,7aRS)-4-Hydroxy-4-m-tolylethynyl-octahydro-indole-1-carboxylic acid ethyl ester
(±)-(3aRS,4SR,7aRS)-4-(4-Fluoro-phenylethynyl)-4-hydroxy-octahydro-indole-1-carboxylic acid ethyl ester
(±)-(3aRS,4SR,7aRS)-4-(3-chlorophenylethynyl)-4-hydroxy-1-methanesulfonyl-octahydro-indole
(±)-(3aRS,7aRS)-4-Phenylethynyl-2,3,3a,6,7,7a-hexahydro-indole-1-carboxylic acid ethyl ester and
(±)-(RS)-4-phenylethynyl-2,3,5,6,7,7a-hexahydro-indole-1-carboxylic acid ethyl ester (±J-CSRSJaRSĴ̂̂-Trifluoro-1-Ĉphenylethynyl̂.S.Sa.ej.ya-hexahydro-indol-1-yl)-ethanone
(±)-(RS)-4-m-Tolylethynyl-2,3,5,6,7,7a-hexahydro-indole-1-carboxylic acid ethyl ester (±)-(3RS,7aRS)-4-m-Tolylethynyl-2,3,3a,6,7,7a-hexahydro-indole-1-carboxylic acid ethyl ester
(±)-(3RS, 7aRS)-4-(4-Chloro-phenylethynyl)-2,3,3a,6,7,7a-hexahydro-indole-1-carboxylic acid ethyl ester
(±)-(3RS, 7aRS)-4-(2-Fluoro-phenylethynyl)-2,3,3a,6,7,7a-hexahydro-indole-1-carboxylic acid ethyl ester
(±)-(3RS,7aRS)-4-(3-Fluoro-phenylethynyl)-2,3,3a,6,7,7a-hexahydro-indole-1-carboxylic acid ethyl ester
(±)-(RS)-4-(3-Fluoro-phenylethynyl)-2,3,5,6,7,7a-hexahydro-indole-1-carboxylic acid ethyl ester
(±)-(3RS,7aRS)-4-(3-Methoxy-phenylethynyl)-2,3,3a,6,7,7a-hexahydro-indole-1-carboxylic acid ethyl ester
(±)-(RS)-4-(3-Methoxy-phenylethynyl)-2,3,5,6,7,7a-hexahydro-indole-1-carboxylic acid ethyl ester
(±)-(3aRS,4RS,7aSR)-4-Hydroxy-4-phenylethynyl-octahydro-isoindole-2-carboxylic acid ethyl ester
(±)-(3aRS,4RS,7aSR)-4-Hydroxy-4-m-tolylethynyl-octahydro-isoindole-2-carboxylic acid ethyl ester
(±)-(3aRS,4RS,7aSR)-4-Hydroxy-4-p-tolylethynyl-octahydro-isoindole-2-carboxylic acid ethyl ester
(±)-(3aRS,4RS,7aSR)-4-(3-Cyano-phenylethynyl)-4-hydroxy-octahydro-isoindole-2-carboxylic acid ethyl ester
(±HSaRŜRSJaSRM-HydroxŷS-methoxy-phenylethynyO-octahydro-isoindolê-carboxylic acid ethyl ester
(±HSaRŜRSJaSRM-CS-Fluoro-phenylethynyl̂-hydroxy-octahydro-isoindolê-carboxylic acid ethyl ester
(±)-(3aRS,4RS,7aSR)-4-Hydroxy-4-phenylethynyl-octahydro-isoindole-2-carboxylic acid tert-butyl ester
(±)-(3aRS,4RS,7aSR)-4-Hydroxy-4-m-tolylethynyl-octahydro-isoindole-2-carboxylic acid tert-butyl ester (±)-(3aRS,4RS,7aSR)-4-Hydroxy-4-m-tolylethynyl-octahydro-isoindole-2-carboxylic acid methyl ester
(±)-(3aRS,4RS,7aSR)-Furan-2-yl-(4-hydroxy-4-m-tolylethynyl-octahydro-isoindol-2-yl)-methanone
(±ĴSaRŜRS.yaSRJ-Cyclopropyl̂-hydroxŷ-m-tolylethynyl-octahydro-isoindol̂-yl)-methanone
(±)-(3aRS,4RS,7aSR)-(4-Hydroxy-4-m-tolylethynyl-octahydro-isoindol-2-yl)-pyridin-3-yl-methanone
(±)-((1SR,3SR)-3-Hydroxy-3-/r7-tolylethynyl-cyclohexyl)-methyl-carbamic acid methyl ester and
(±)-((1RS,3SR)-3-hydroxy-3-/n-tolylethynyl-cyclohexyl)-methyl-carbamic acid methyl ester
(±)-(1RS,3SR)-((3-Hydroxy-3-/n-tolylethynyl-cyclohexyl)-(4-methoxy-benzyl)-carbamic acid ethyl ester
(±)-(1RS,3RS)-((3-Hydroxy-3-/r7-tolylethynyl-cyclohexyl)-(4-methoxy-benzyl)-carbamic acid ethyl ester
(±)-[(1RS,3SR)-3-Hydroxy-3-(3-methoxy-phenylethynyl)-5,5-dimethyl-cyclohexyl]-methyl-carbamic acid methyl ester
(±)-(1RS,3SR)-(3-Hydroxy-5,5-dimethyl-3-/r7-tolylethynyl-cyclohexyl)-methyl-carbamic acid methyl ester
(±)-[(1RS,3 SR)-3-(3-Fluoro-phenylethynyl)-3-hydroxy-5,5-dimethyl-cyclohexyl]-methyl-carbamic acid methyl ester
(±)-[(1RS,3RS)-3-(3-Fluoro-phenylethynyl)-3-hydroxy-cyclohexyl]-methyl-carbamic acid methyl ester
(±)-[(1RS,3 SR)-3-(3-Fluoro-phenylethynyl)-3-hydroxy-cyclohexyl]-methyl-carbamic acid methyl ester
(±)-[(1RS,3RS)-3-Hydroxy-3-(3-methoxy-phenylethynyl)-cyclohexyl]-methyl-carbamic acid methyl ester
(±)-[(1RS,3 SR)-3-Hydroxy-3-(3-methoxy-phenylethynyl)-cyclohexyl]-methyl-carbamic acid methyl ester
(±)-[(1RS,3RS)-3-(3-Chloro-phenylethynyl)-3-hydroxy-cyclohexyl]-methyl-carbamic acid methyl ester
(±)-[(1RS,3 SR)-3-(3-Chloro-phenylethynyl)-3-hydroxy-cyclohexyl]-methyl-carbamic acid methyl ester (±)-(1RS,3RS)—N-(3-hydroxy-3-m-tolylethynyl-cyclohexyl)-acetamide
(±)-(1RS,3 SR)—N-(3-hydroxy-3-m-tolylethynyl-cyclohexyl)-acetamide
(±)-(1RS,3 RS)-(3-Hydroxy-3-m-tolylethynyl-cyclohexyl)-carbamic acid ethyl ester
(±)-(1RS,3 SR)-(3-Hydroxy-3-m-tolylethynyl-cyclohexyl)-carbamic acid ethyl ester
(±)-(1RS,3RS)-[3-(3-Fluoro-phenylethynyl)-3-hydroxy-cyclohexyl]-carbamic acid ethyl ester
(±)-(1RS,3SR)-[3-(3-Fluoro-phenylethynyl)-3-hydroxy-cyclohexyl]-carbamic acid ethyl ester
(±)-(1RS,3RS)-[3-(3-Methoxy-phenylethynyl)-3-hydroxy-cyclohexyl]-carbamic acid ethyl ester
(±)-(1RS,3RS)—N-[3-(3-Fluoro-phenylethynyl)-3-hydroxy-cyclohexyl]-acetamide.
(±)-(1RS,3 SR)—N-[3-(3-Fluoro-phenylethynyl)-3-hydroxy-cyclohexyl]-acetamide
(±)-(1RS,3SR)-[3-Hydroxy-3-(3-methoxy-phenylethynyl)-cyclohexyl]-carbamic acid ethyl ester
(±)-(1RS,3RS)—N-[3-Hydroxy-3-(3-methoxy-phenylethynyl)-cyclohexyl]-acetamide
(±)-(1RS,3SR)—N-[3-Hydroxy-3-(3-methoxy-phenylethynyl)-cyclohexyl]-acetamide.
(±)-(1RS,3RS)-[3-Hydroxy-3-(3-methoxy-phenylethynyl)-cyclohexyl]-carbamic acid tert-butyl ester
(±)-(1RS,3SR)-[3-Hydroxy-3-(3-methoxy-phenylethynyl)-cyclohexyl]-carbamic acid tert-butyl ester
(±)-(1RS,3RS)-(3-Hydroxy-3-m-tolylethynyl-cyclohexyl)-carbamic acid tert-butyl ester (±)-(1 RS,3SR)-(3-Hydroxy-3-m-tolylethynyl-cyclohexyl)-carbamic acid tert-butyl ester (±)-(1RS,3RS)-(3-(3-Fluoro-phenylethynyl)-3-hydroxy-cyclohexyl]-carbamic acid tert-butyl ester
(±)-(1RS,3SR)-(3-(3-Fluoro-phenylethynyl)-3-hydroxy-cyclohexyl]-carbamic acid tert-butyl ester
(±)-(1RS,3RS)-[3-(3-Fluoro-phenylethynyl)-3-hydroxy-cyclohexyl]-carbamic acid methyl ester (±)-(1RS,3SR)-[3-(3-Fluoro-phenylethynyl)-3-hydroxy-cyclohexyl]-carbamic acid methyl ester (±)-(3-Phenylethynyl-cyclohex-2-enyl)-carbamic acid ethyl ester and (±)-3-phenylethynyl-cyclohex-3-enyl)-carbamic acid ethyl ester
(±)-Methyl-(3-phenylethynyl-cyclohex-3-enyl)-carbamic acid ethyl ester
(±)-(4aRS,5RS,8aSR)-5-Hydroxy-5-phenylethynyl-octahydro-quinoline-1-carboxylic acid ethyl ester
(±)-[(4aRS,5SR,8aSR)-5-(3-Chloro-phenylethynyl)-5-hydroxy-octahydro-quinolin-1-yl]-furan-2-yl-methanone (±)-[(4aRS,5RS,8aSR)-5-(3-Chloro-phenylethynyl)-5-hydroxy-octahydro-quinolin-1-yl]-furan-2-yl-methanone
(±)-(4aRS,5RS,8aSR)-5-(3-Chloro-phenylethynyl)-5-hydroxy-octahydro-quinoline-1-carboxylic acid tert-butyl ester
(±)-[(4aRS,5SR,8aSR)-5-(3-Chloro-phenylethynyl)-5-hydroxy-octahydro-quinolin-1-yl]-morpholin-4-yl-methanone
(±)-[(4aRS,5SR,8aSR)-5-(3-chloro-phenylethynyl)-5-hydroxy-octahydro-quinolin-1-yl]-(4-methyl-piperazin-1-yl)-methanone
(±)-(4aRS,5RS,8aSR)-5-(3-chloro-phenylethynyl)-5-hydroxy-octahydro-quinoline-1-carboxylic acid ethyl ester and (±)-(4aRS,5SR,8aSR)-5-(3-chloro-phenylethynyl)-5-hydroxy-octahydro-quinoline-1-carboxylic acid ethyl ester
(±)-(4aRS,5SR,8aSR)-5-Hydroxy-5-m-tolylethynyl-octahydro-quinoline-1-carboxylic acid ethyl ester
(±)-(4aRS,5RS,8aSR)-5-Hydroxy-5-m-tolylethynyl-octahydro-quinoline-1-carboxylic acid ethyl ester.

In a further embodiment, the mGluR modulator is a compound of the formula (V):

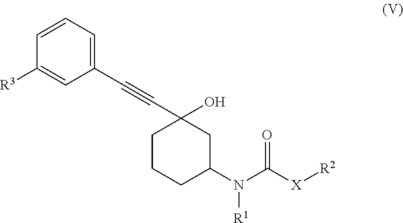

wherein
R¹ represents hydrogen or alkyl;
R² represents an unsubstituted or substituted heterocycle or
R² represents an unsubstituted or substituted aryl;
R³ represents alkyl or halogen;
X represents a single bond or an alkandiyl-group, optionally interrupted by one or more oxygen atoms or carbonyl groups or carbonyloxy groups in free base or acid addition salt form.

Exemplary compounds of formula (V) include:
Furan-3-carboxylic acid [(1S,3S)-3-(3-chloro-phenylethynyl)-3-hydroxy-cyclohexyl]-amide Furan-2-carboxylic acid [(1R,3R)-3-(3-chloro-phenylethynyl)-3-hydroxy-cyclohexyl]-amide Furan-2-carboxylic acid [(1S,3S)-3-(3-chloro-phenylethynyl)-3-hydroxy-cyclohexyl]-amide 3H-Imidazole-4-carboxylic acid [(1R,3R)-3-(3-chloro-phenylethynyl)-3-hydroxy-cyclohexyl]-amide
3H-Imidazole-4-carboxylic acid [(1S,3S)-3-(3-chloro-phenylethynyl)-3-hydroxy-cyclohexyl]-amide
4H-[1,2,4]Triazole-3-carboxylic acid [(1R,3R)-3-(3-chloro-phenylethynyl)-3-hydroxy-cyclohexyl]-amide
4H-[1,2,4]Triazole-3-carboxylic acid [(1S,3S)-3-(3-chloro-phenylethynyl)-3-hydroxy-cyclohexyl]-amide
2-Methyl-furan-3-carboxylic acid [(±)-(1R,3R)-3-(3-chloro-phenylethynyl)-3-hydroxy-cyclohexyl]-amide
N-[(±)-(1R,3R)-3-(3-Chloro-phenylethynyl)-3-hydroxy-cyclohexyl]-3,4-difluoro-benzamide
Benzo[1,3]dioxole-2-carboxylic acid [(±)-(1R,3R)-3-(3-chloro-phenylethynyl)-3-hydroxy-cyclohexyl]-amide
5-Methyl-pyrazine-2-carboxylic acid [(±)-(1R,3R)-3-(3-chloro-phenylethynyl)-3-hydroxy-cyclohexyl]-amide
Quinoxaline-2-carboxylic acid [(±)-(1R,3R)-3-(3-chloro-phenylethynyl)-3-hydroxy-cyclohexyl]-amide
Benzofuran-2-carboxylic acid [(±)-(1R,3R)-3-(3-chloro-phenylethynyl)-3-hydroxy-cyclohexyl]-amide
Benzooxazole-2-carboxylic acid [(±)-(1R,3R)-3-(3-chloro-phenylethynyl)-3-hydroxy-cyclohexyl]-amide
2,5-Dimethyl-furan-3-carboxylic acid [(±)-(1R,3R)-3-(3-chloro-phenylethynyl)-3-hydroxy-cyclohexyl]-amide
(R,S)-Tetrahydro-furan-3-carboxylic acid [(±)-(1R,3R)-3-(3-chloro-phenylethynyl)-3-hydroxy-cyclohexyl]-amide
Furan-3-carboxylic acid ((1R.SRJ-S-hydroxy-S-m-tolylethynyl-cyclohexyO-amide
Furan-3-carboxylic acid ((1S,3S)-3-hydroxy-3-m-tolylethynyl-cyclohexyl)-amide Furan-3-carboxylic acid ((±)-(1R.SRJ-S-hydroxy-S-m-tolylethynyl-cyclohexyO-amide
Furan-2-carboxylic acid ((1R,3R)-3-hydroxy-3-m-tolylethynyl-cyclohexyl)-amide
Furan-2-carboxylic acid ((1S,3S)-3-hydroxy-3-m-tolylethynyl-cyclohexyl)-amide
Furan-2-carboxylic acid ((±)-(1R,3R)-3-hydroxy-3-m-tolylethynyl-cyclohexyl)-amide Isoxazole-5-carboxylic acid ((1R,3R)-3-hydroxy-3-m-tolylethynyl-cyclohexyl)-amide Isoxazole-5-carboxylic acid ((1S,3S)-3-hydroxy-3-m-tolylethynyl-cyclohexyl)-amide Isoxazole-5-carboxylic acid ((±)-(1 R,3R)-3-hydroxy-3-m-tolylethynyl-cyclohexyl)-amide
5-Methyl-pyrazine-2-carboxylic acid ((±)-(1R,3R)-3-hydroxy-3-m-tolylethynyl-cyclohexyl)-amide 4H-[1,2,4]Triazole-3-carboxylic acid ((±)-(1R,3R)-3-hydroxy-3-m-tolylethynyl-cyclohexyl)-amide
3H-Imidazole-4-carboxylic acid ((±)-(1R,3R)-3-hydroxy-3-m-tolylethynyl-cyclohexyl)-amide
Tetrahydro-pyran-4-carboxylic acid ((±)-(1R,3R)-3-hydroxy-3-m-tolylethynyl-cyclohexyl)-amide
1-Methyl-1H-imidazole-4-carboxylic acid ((±)-(1R,3R)-3-hydroxy-3-m-tolylethynyl-cyclohexyl)-amide
(R,S)-Tetrahydro-furan-2-carboxylic acid ((±)-(1R,3R)-3-hydroxy-3-m-tolylethynyl-cyclohexyl)-amide
(R,S)-Tetrahydro-furan-3-carboxylic acid ((±)-(1R,3R)-3-hydroxy-3-m-tolylethynyl-cyclohexyl)-amide
Furan-3-carboxylic acid [(1R,3R)-3-(3-fluoro-phenylethynyl)-3-hydroxy-cyclohexyl]-amide Furan-3-carboxylic acid [(1S,3S)-3-(3-fluoro-phenylethynyl)-3-hydroxy-cyclohexyl]-amide Furan-2-carboxylic acid [(1R,3R)-3-(3-fluoro-phenylethynyl)-3-hydroxy-cyclohexyl]-amide Furan-2-carboxylic acid [(1S,3S)-3-(3-fluoro-phenylethynyl)-3-hydroxy-cyclohexyl]-amide 3H-Imidazole-4-carboxylic acid [(±)-(1R,3R)-3-(3-fluoro-phenylethynyl)-3-hydroxy-cyclohexyl]-amide
N-[(1S,3S)-3-(3-Chloro-phenylethynyl)-3-hydroxy-cyclohexyl]-3,4-difluoro-benzamide N-[(1 R,3R)-3-(3-Chloro-phenylethynyl)-3-hydroxy-cyclohexyl]-3,4-difluoro-benzamide Pyridine-2-carboxylic acid [(1S,3S)-3-(3-chloro-phenylethynyl)-3-hydroxy-cyclohexyl]-amide Pyridine-2-carboxylic acid [(1R,3R)-3-(3-chloro-phenylethynyl)-3-hydroxy-cyclohexyl]-amide N-[(1S,3S)-3-(3-Chloro-phenylethynyl)-3-hydroxy-cyclohexyl]-nicotinamide
N-[(1R,3R)-3-(3-Chloro-phenylethynyl)-3-hydroxy-cyclohexyl]-nicotinamide Benzo[1,3]dioxole-2-carboxylic acid [(1S,3S)-3-(3-chloro-phenylethynyl)-3-hydroxy-cyclohexyl]-amide
5-Methyl-pyrazine-2-carboxylic acid [(1S,3S)-3-(3-chloro-phenylethynyl)-3-hydroxy-cyclohexyl]-amide
2-Methyl-furan-3-carboxylic acid [(1S,3S)-3-(3-chloro-phenylethynyl)-3-hydroxy-cyclohexyl]-amide
(R)-Tetrahydro-furan-2-carboxylic acid [(1R,3R)-3-(3-chloro-phenylethynyl)-3-hydroxy-cyclohexyl]-amide
(S)-Tetrahydro-furan-2-carboxylic acid [(1R,3R)-3-(3-chloro-phenylethynyl)-3-hydroxy-cyclohexyl]-amide
Isoxazole-5-carboxylic acid [(1S,3S)-3-(3-chloro-phenylethynyl)-3-hydroxy-cyclohexyl]-amide 5-Methyl-pyrazine-2-carboxylic acid [(1R,3R)-3-(3-chloro-phenylethynyl)-3-hydroxy-cyclohexyl]-amide
2-Methyl-furan-3-carboxylic acid [(1R,3R)-3-(3-chloro-phenylethynyl)-3-hydroxy-cyclohexyl]-amide
Isoxazole-5-carboxylic acid [(1R,3R)-3-(3-chloro-phenylethynyl)-3-hydroxy-cyclohexyl]-amide 5-Chloro-furan-2-carboxylic acid [(1R,3R)-3-(3-chloro-phenylethynyl)-3-hydroxy-cyclohexyl]-amide
5-Chloro-furan-2-carboxylic acid [(1S,3S)-3-(3-chloro-phenylethynyl)-3-hydroxy-cyclohexyl]-amide
(S)-Tetrahydro-furan-3-carboxylic acid [(1R,3R)-3-(3-chloro-phenylethynyl)-3-hydroxy-cyclohexyl]-amide
(R)-Tetrahydro-furan-3-carboxylic acid [(1R,3R)-3-(3-chloro-phenylethynyl)-3-hydroxy-cyclohexyl]-amide
N-[(1S,3S)-3-(3-Chloro-phenylethynyl)-3-hydroxy-cyclohexyl]-isonicotinamide
N-[(1R,3R)-3-(3-Chloro-phenylethynyl)-3-hydroxy-cyclohexyl]-isonicotinamide
3,5-Difluoro-pyridine-2-carboxylicacid [(1R,3R)-3-(3-chloro-phenylethynyl)-3-hydroxy-cyclohexyl]-amide
3,5-Difluoro-pyridine-2-carboxylicacid [(1S,3S)-3-(3-chloro-phenylethynyl)-3-hydroxy-cyclohexyl]-amide
6-Methyl-pyridine-2-carboxylic acid [(1S,3S)-3-(3-chloro-phenylethynyl)-3-hydroxy-cyclohexyl]-amide 6-Methyl-pyridine-2-carboxylic acid [(1R,3R)-3-(3-chloro-phenylethynyl)-3-hydroxy-cyclohexyl]-amide
S-Chloro-pyridinê-carboxylic acid [(1R,3R)-3-(3-chloro-phenylethynyl)-3-hydroxy-cyclohexyl]-amide
δ-Chloro-pyridinê-carboxylic acid [(1S,3S)-3-(3-chloro-phenylethynyl)-3-hydroxy-cyclohexyl]-amide
δ-Chloro-pyridinê-carboxylic acid [(1R,3R)-3-(3-chloro-phenylethynyl)-3-hydroxy-cyclohexyl]-amide
6-Chloro-pyridine-2-carboxylic acid [(1S,3S)-3-(3-chloro-phenylethynyl)-3-hydroxy-cyclohexyl]-amide
5-Chloro-1-methyl-1H-pyrrole-2-carboxylic acid [(1R,3R)-3-(3-chloro-phenylethynyl)-3-hydroxy-cyclohexyl]-amide
5-Chloro-1-methyl-1H-pyrrole-2-carboxylic acid [(1S,3S)-3-(3-chloro-phenylethynyl)-3-hydroxy-cyclohexyl]-amide
5-Chloro-1H-pyrrole-2-carboxylic acid [(1R,3R)-3-(3-chloro-phenylethynyl)-3-hydroxy-cyclohexyl]-amide
5-Chloro-1H-pyrrole-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-4-dimethyl amino-benzamide
1H-Pyrrole-3-carboxylic acid [(1S,3S)-3-(3-chloro-phenylethynyl)-3-hydroxy-cyclohexyl]-amide
N-[(1S,3S)-3-(3-chloro-phenylethynyl)-3-hydroxy-cyclohexyl]-4-methyl-benzamide
N-[(1S,3S)-3-(3-Chloro-phenylethynyl)-3-hydroxy-cyclohexyl]-4-methyl-benzamide
N-[(1S,3S)-3-(3-Chloro-phenylethynyl)-3-hydroxy-cyclohexyl]-3-fluoro-benzamide
N-[(1S,3S)-3-(3-Chloro-phenylethynyl)-3-hydroxy-cyclohexyl]-2-ethyl-butyramide
N-[(1S,3S)-3-(3-Chloro-phenylethynyl)-3-hydroxy-cyclohexyl]-4-(2,5-dimethoxy-phenyl)-4-oxo-butyramide
2-(2-Benzyloxy-ethoxy)-N-[(1S,3S)-3-(3-chloro-phenylethynyl)-3-hydroxy-cyclohexyl]-acetamide
N-[(1S,3S)-3-(3-Chloro-phenylethynyl)-3-hydroxy-cyclohexyl]-2-phenyl-acetamide
N-[(1S,3S)-3-(3-Chloro-phenylethynyl)-3-hydroxy-cyclohexyl]-3-(1H-indol-4-yl)-propionamide 2-Benzo[1,3]dioxol-5-yl-N-[(1S,3S)-3-(3-chloro-phenylethynyl)-3-hydroxy-cyclohexyl]-acetamide
N-[(1S,3S)-3-(3-Chloro-phenylethynyl)-3-hydroxy-cyclohexyl]-2-phenoxy-propionamide
N-[(1S,3S)-3-(3-Chloro-phenylethynyl)-3-hydroxy-cyclohexyl]-2-(2-fluoro-phenyl)-acetamide
5-Hydroxy-1H-indole-2-carboxylic acid [(1S,3S)-3-(3-chloro-phenylethynyl)-3-hydroxy-cyclohexyl]-amide
1-Methyl-1H-pyrrole-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-terephthalamic acid methyl ester
N-[(1S,3S)-3-(3-Chloro-phenylethynyl)-3-hydroxy-cyclohexyl]-2-(2-trifluoromethoxy-phenyl)-acetamide
5-Chloro-N-[(1S,3S)-3-(3-chloro-phenylethynyl)-3-hydroxy-cyclohexyl]-2-hydroxy-benzamide
N-[(1S,3S)-3-(3-Chloro-phenylethynyl)-3-hydroxy-cyclohexyl]-4-hydroxy-benzamide
N-[(1S,3S)-3-(3-Chloro-phenylethynyl)-3-hydroxy-cyclohexyl]-2-hydroxy-benzamide
4-Amino-N-[(1S,3S)-3-(3-chloro-phenylethynyl)-3-hydroxy-cyclohexyl]-benzamide
4-Amino-5-chloro-N-[(1S,3S)-3-(3-chloro-phenylethynyl)-3-hydroxy-3-Amino-4-chloro-N-[(1 S,3S)-3-(3-chloro-phenylethynyl)-3-hydroxy-cyclohexyl]-benzamide
3-Amino-N-[(1S,3S)-3-(3-chloro-phenylethynyl)-3-hydroxy-cyclohexyl]-4-methyl-benzamide
2-Amino-N-[(1S,3S)-3-(3-chloro-phenylethynyl)-3-hydroxy-cyclohexyl]-nicotinamide
N-[(1S,3S)-3-(3-Chloro-phenylethynyl)-3-hydroxy-cyclohexyl]-4-hydroxy-3-methoxy-benzamide
N-[(1S,3S)-3-(3-Chloro-phenylethynyl)-3-hydroxy-cyclohexyl]-2-fluoro-benzamide
N-[(1S,3S)-3-(3-Chloro-phenylethynyl)-3-hydroxy-cyclohexyl]-4-methanesulfonyl-benzamide
Pyridine-2-carboxylic acid [(1S,3S)-3-(3-chloro-phenylethynyl)-3-hydroxy-cyclohexyl]-amide
3-Amino-pyrazine-2-carboxylic acid [(1S,3S)-3-(3-chloro-phenylethynyl)-3-hydroxy-cyclohexyl]-amide
6-Amino-N-[(1S,3S)-3-(3-chloro-phenylethynyl)-3-hydroxy-cyclohexyl]-nicotinamide
4-(4-Amino-benzoylamino)-benzoic acid [(1R,3R)-3-(3-chloro-phenylethynyl)-3-hydroxy-cyclohexyl]-amide
2,6-Dioxo-1,2,3,6-tetrahydro-pyrimidine-4-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-isonicotinamide 3-Chloro-N-[(1S,3S)-3-(3-chloro-phenylethynyl)-3-hydroxy-cyclohexyl]-benzamide
N-[(1S,3S)-3-(3-Chloro-phenylethynyl)-3-hydroxy-cyclohexyl]-2,3-dimethoxy-benzamide
N-[(1S,3S)-3-(3-Chloro-phenylethynyl)-3-hydroxy-cyclohexyl]-4-oxo-4-phenyl-butyramide
2-Chloro-N-[(1S,3S)-3-(3-chloro-phenylethynyl)-3-hydroxy-cyclohexyl]-nicotinamide
5-Bromo-N-[(1S,3S)-3-(3-chloro-phenylethynyl)-3-hydroxy-cyclohexyl]-nicotinamide Isoquinoline-1-carboxylic acid [(1S,3S)-3-(3-chloro-phenylethynyl)-3-hydroxy-cyclohexyl]-amide
Pyrazine-2-carboxylic acid [(1S,3S)-3-(3-chloro-phenylethynyl)-3-hydroxy-cyclohexyl]-amide
3-Benzoyl-pyridine-2-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-methyl-nicotinamide
Quinoxaline-2-carboxylic acid [(1S,3S)-3-(3-chloro-phenylethynyl)-3-hydroxy-cyclohexyl]-amide
Pyridazine-4-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-methylsulfanyl-nicotinamide
N-[(1S,3S)-3-(3-Chloro-phenylethynyl)-3-hydroxy-cyclohexyl]-4-trifluoromethyl-nicotinamide
2-Chloro-N-[(1S,3S)-3-(3-chloro-phenylethynyl)-3-hydroxy-cyclohexyl]-isonicotinamide
2-Chloro-N-[(1S,3S)-3-(3-chloro-phenylethynyl)-3-hydroxy-cyclohexyl]-6-methyl-nicotinamide
6-Chloro-N-[(1S,3S)-3-(3-chloro-phenylethynyl)-3-hydroxy-cyclohexyl]-nicotinamide
2-Chloro-N-[(1S,3S)-3-(3-chloro-phenylethynyl)-3-hydroxy-cyclohexyl]-6-methyl-isonicotinamide
N-[(1S,3S)-3-(3-Chloro-phenylethynyl)-3-hydroxy-cyclohexyl]-2-(4,5-dimethoxy-3-oxo-1,3-dihydro-isobenzofuran-1-yl)-acetamide
1̂.δ.β-Tetrahydro-cyclopentapyrazole-5-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-3-(1H-indol-2-yl)-propionamide
6-[(1S,3S)-3-(3-Chloro-phenylethynyl)-3-hydroxy-cyclohexylcarbamoyl]-pyridine-2-carboxylic acid isopropyl ester
Quinoline-6-carboxylic acid [(1S,3S)-3-(3-chloro-phenylethynyl)-3-hydroxy-cyclohexyl]-amide
5-Methyl-isoxazole-4-carboxylic acid [(1S,3S)-3-(3-chloro-phenylethynyl)-3-hydroxy-cyclohexyl]-amide Benzofuran-3-carboxylic acid [(1S,3S)-3-(3-chloro-phenylethynyl)-3-hydroxy-cyclohexyl]-amide
N-[(1S,3S)-3-(3-Chloro-phenylethynyl)-3-hydroxy-cyclohexyl]-2-(2-methoxy-phenoxy)-acetamide.

In a further embodiment, the mGluR modulator is a compound of the formula (VI)

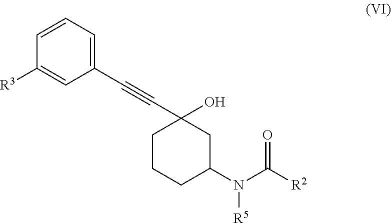

wherein
R¹ represents hydrogen or alkyl;
R R² represents an unsubstituted or substituted heterocycle or
R² represents an unsubstituted or substituted aryl;
R³ represents alkyl or halogen;
in free base or acid addition salt form.

Further examples mGluR5 antagonists include compounds of the formula (I) as defined in WO 2004/014881 and compounds of the formula (I) as defined in WO 2007/021575; the contents of these publications are incorporated herein by reference.

### Biomarkers

The extent of FMR1 gene methylation, the lack of the presence of FMR1 mRNA expression and the lack of the presence of FMR1 protein in a sample from an individual having FXS can individually, or in any combination, serve as biomarkers to predict responsiveness of that individual to treatment with an mGluR5 antagonist.

The presence of these biomarkers can be determined in a sample from an individual of interest. The sample can be any sample including a fluid sample such as blood, a cell sample such as buccal cells, or a tissue sample such as skin or a hair follicle.

As used herein, “predicting” indicates that the methods described herein provide information to enable a health care provider to determine the likelihood that an individual having FXS will respond to mGluR5 treatment. Following a positive determination of the relevant biomarker(s) in a sample of interest, the individual will be administered an mGluR5 antagonist.

### FMR1 Promoter—Methylation Analysis

The extent of FMR1 gene methylation is indicative as to whether a patient will respond therapeutically to an mGluR5 antagonist. Specifically, if an individual is determined to have all, or predominately all, of the FMR1 gene region of interest methylated, then that individual is determined to be an individual that will respond to treatment with an mGluR5 antagonist.

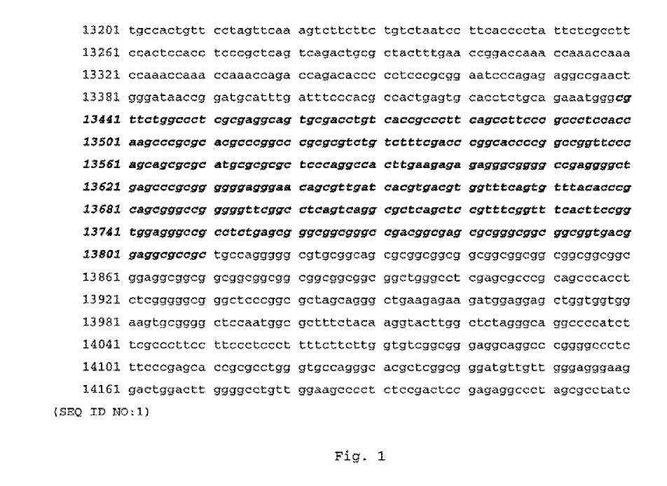

The sequence of the FMR1 gene is known in the art (GenBank L29074 L38501) (Nucleic Acids Res. 2002 Jul. 15; 30(14):3278-85) (Hum Mol Genet. 2010 Apr. 15; 19(8):1618-32. Epub 2010 Jan. 29). FIG. 1 depicts the FMR1 promoter region and 5′-UTR of the FMR1 gene. Also shown in FIG. 1 is the sequence and location of FREE1/2, the classical CpG island and CGG repeats in the FMR1 gene. The sequence numbering is from GenBank L29074 L38501. The classical CpG island with 52 CpG sites is located in the position from 13439 to 13809. The CGG repeats are located in the position from 13833 to 13892. The FREE1 is located in the position from 13227 to 13439 (upstream of the classical CpG island). The FREE2 is located in the position from 13951 to 14199 (downstream of the CGG repeats).

The FMR1 gene that is analyzed for its methylation status according to the present invention can be of any length as long as it includes at least one CpG site. In one example, the FMR1 gene region being analyzed is the FMR1 classical CpG island with 52 CpG sites (see FIG. 1; this region is bolded in the figure; SEQ ID NO:1). In another example, the upstream region (FREE1) and/or the down stream region (FREE2) of the FMR1 classical CpG island is analyzed. The methylation of FREE1 and FREE2 is highly correlated with the FMR1 classical CpG island (Hum Mol Genet. 2010 Apr. 15; 19(8):1618-32. Epub 2010 Jan. 29.).

In yet another example, the CGG repeats that are located in the 5′-UTR of the FMR1 gene are analyzed for their methylation status and can be used to determine if an individual is an mGluR5 responder.

In another example, a portion of the FMR1 promoter region is analyzed for its methylation status. In a particular example, the FMR1 promoter region is one with 22 CpG sites and has the nucleotide sequence of SEQ ID NO:2 (shown below; CpG sites are shown in bold and underlined).

(SEQ ID NO: 2)
5GCCACTGAGTGCACCTCTGCAGAAATGGGCGTTCTGGCCCTCGCGAG
GCAGTGCGACCTGTCACCGCCCTTCAGCCTTCCCGCCCTCCACCAAGC
CCGCGCACGCCCGGCCCGCGCGTCTGTCTTTCGACCCGGCACCCCGGC
CGGTTCCCAGCAGCGCGCATGCGCGCGCTCCCAGGCCACTTGAAGAGA
GAGGG-3.

In another example of a portion of the FMR1 promoter region that can be analyzed is SEQ ID NO:3 that has 15 CpGs.

(SEQ ID NO: 3;
CpG sites are shown in bold and underlined)
5TGCAGAAATGGGCGTTCTGGCCCTCGCGAGGCAGTGCGACCTGTCAC
CGCCCTTCAGCCTTCCCGCCCTCCACCAAGCCCGCGCACGCCCGGCCC
GCGCGTCTGTCTTTCGACCCGGCAC-3

The extent of methylation of the FMR1 gene region is determined by detecting for the presence, or absence, or the level, of a methyl group modification in the cytosine of that FMR1 gene region.

Various methods can be used to determine the methylation status of an individual of interest. Using a qualitative assay such as MSP an individual is determined to be a responder when only methylated FMR1 is detected in the region of interest in the biological sample. Such a patient is also referred to herein as an individual having “all” of the FMR1 gene methylated in the biological sample or as “fully methylated”. For clarity, an individual assigned to be a responder using such a qualitative assay is an individual where no unmethylated FMR1 is detected in the region of interest. In contrast, an individual that has an FMR1 gene region that is “partially methylated” refers to an individual having both methylated and unmethylated FMR1 present in the gene region of interest (e.g., the gene region has a level of methylation of less than 80%) and such an individual is an mGluR5 non-responder.

Another method useful in the method of the invention is a quantitative assay method such as methylation-sensitive restriction enzyme digestion combined with quantitative PCR, matrix-assisted laser desorption/ionization time-to-flight mass spectrometry (MALDI-TOF-MS), real-time PCR (methyl light). An individual is determined to be an mGluR5 responder if that individual has all, or predominately all, of the FMR1 gene methylated. As used herein, “predominately all” is when the methylated FMR1 gene region in the biological sample shows a level of methylation, e.g., 99.5% or higher, e.g., 99.6, 99.7, 99.8, or 99.9% or when the methylated FMR1 gene region in the biological sample shows a delta Ct of 8.0 or higher, e.g., 8.5. Using a quantitative assay method as described herein, an individual can also be referred to as having a “fully methylated” FMR1 gene region of interest when “all, or predominately all” of the FMR1 gene region of interest is methylated. Such an individual is an mGluR5 responder.

The invention is not limited by the types of assays used to assess the extent of methylation of the FMR1 gene region in the sample. Indeed, any assay that can be employed to determine the methylation status of a gene can be employed for the purposes of the present invention. Examples of types of assays used to assess the methylation pattern include, but are not limited to:
(i) methylation-sensitive restriction enzyme digestion combined with at least one of: hybridization, quantitative PCR, restriction landmark genomic scanning (RLGS), or array-based profiling of reference-independent methylation status (aPRIMEs);
(ii) bisulfate DNA modification combined with at least one of: methylation specific PCR (MS-PCR), quantitative methylation specific PCR (qMS-PCR), probe-based methylation specific PCR, pyrosequencing, cloning/sequencing, MS-nested PCR, quantitative analysis of methylated alleles (QUAMA), heavy methyl detection, methylation-sensitive high resolution melting (MS-HRM), methyl-binding (MB)-PCR, PCR and deoxyribonucleoside monophosphate (dNMP) analysis, or methylation-dependent fragment separation (MDFS);
(iii) total hydrolysis followed by high-performance liquid chromatography (HPLC);
(iv) combination of methylated-DNA precipitation and methylation-sensitive restriction enzymes (COMPARE-MS);
(v) combined bisulfite restriction analysis (COBRA); direct or indirect detection of methylated DNA molecules in a nano transistor or other electronic based device; and;
(vi) methyl-BEAMing (beads, emulsion, amplification and magnetics) technology.

In one example, the extent of methylation can be determined using Methylation Specific PCR (MSP). MSP is a bisulfite conversion based PCR technique which can be used to determine DNA CpG methylation. MSP involves the initial modification of DNA by sodium bisulfite which converts all unmethylated, but not methylated, cytosines to uracil. The DNA is then amplified with two pairs of primers specific for methylated DNA and unmethylated DNA, respectively, and the methylation status determined. The primers typically include at least two CpG sites. The MSP methods are described in U.S. Pat. No. 5,786,146; U.S. Pat. No. 6,017,704; U.S. Pat. No. 6,200,756; and U.S. Pat. No. 6,265,171; the entire contents of each of which is incorporated herein by reference. In one example, using the MSP assay, an individual would be assigned as an mGluR5 responder when the methylated FMR1 is detected by the primers specific for methylated DNA and unmethylated FMR1 is not detected by the primers specific for unmethylated DNA in the region of interest of the FMR1 gene.

In another example, the extent of methylation can be determined using a method that includes an amplification process such as quantitative PCR (qPCR) in the FMR1 gene region of interest. Various different qPCR methods which detect methylation are known in the art and include HeavyMethyl or Methylight. Using the HeavyMethyl method, the FMR1 gene region is initially modificated by sodium bisulfite. The DNA is then contacted with non-extendable oligonucleotide blockers that provide specificity by binding to bisulfite-treated DNA in a methylation-specific manner. The DNA is then contacted with a primer set that has binding sites that overlap with non-extendable oligonucleotide blockers. When the blocker is bound, the primer cannot bind and therefore no amplicon is generated. Conversely, if the blocker is not bound, the primers can bind and generate an amplicon (Cottrell et al. Nucleic Acids Res. 2004; 32(1), 2004).

Using the MethyLight method, the FMR1 gene region of interest is initially modified by sodium bisulfite. The gene region is then amplified using PCR primers that hybridize to regions containing no CpG dinucleotides. By using fluorescent-labelled probes that hybridize only to sequences resulting from bisulfite conversion of unmethylated DNA, (or alternatively to methylated sequences that are converted), fluorescent probe detection can indicate methylation status of sequences where the probes hybridize.

Methods for detecting methylation of a region of interest by cutting the DNA with a methylation-sensitive restriction enzyme and subsequently selectively identifying and/or analyzing the cut or uncut DNA are known in the art. The method can encompass amplifying intact DNA after restriction enzyme digestion see, e.g., U.S. patent application Ser. Nos. 10/971,986; 11/071,013; and 10/971,339.

In one example, the method of the invention includes digesting the FMR1 gene promoter region with a methylation sensitive restriction enzyme and amplifying up the region of interest. The methylation status of the DNA can be determined by detecting for the presence of an amplifiable product. Only DNA that was not cleaved by the restriction enzyme will be amplified. A methylation sensitive restriction enzyme can be for example, McrBC, which includes CG as part of its recognition site and can cleave when the C is methylated. Additionally the sample can be contacted with a restriction enzyme which includes CG as part of its recognition site and can cleave only when the C is unmethylated. Following digestion, the desired FMR1 promoter region can be amplified by real-time PCR using a forward/reverse oligonucleotide and a detecting probe. The probe for detecting nucleic acid sequence typically has a fluorescent reporter or fluorophore such as 6-carboxyfluorescein (FAM) and tetrachlorofluorescin (TET) and a quencher such as tetramethylrhodamine (TAMRA) or black hole quencher (BHQ) covalently attached to its 5′ and 3′ ends, respectively.

Illustrative examples of primers that can be used in this assay include Forward Primer (F1): TGCAGAAATGGGCGTTCT (SEQ ID NO:4); Reverse Primer (R1): GTGCCGGGTCGAAAGAC (SEQ ID NO:5) and Probe (P1): Dye-CTGAAGGGCGGTGACAGGTCG (SEQ ID NO:6)-Quencher (e.g. Dye-FAM; Quencher-BHQ1). Using this method a clinical cut-off Region constituting a deltaCt is determined (difference in PCR Cycle threshold values between McrBC and Untreated channels). The Delta Ct values can also be expressed as percent methylation using a mathematical algorithm (e.g., see Holemon et al., Biotechniques 43:683-693, 2007). In one example, a Delta Ct of 8.0 or above (which corresponds to a sample having an FMR1 gene region having 99.95%, or above, methylation) is determinative of a patient which has all, or predominately all, of the FMR1 gene region methylated and is therefore an mGluR5 responder.

The methods described above can be used with a methylation analyzer. Typically, the method includes determining the extent of FMR1 methylation in the sample, transforming the results into a computer readable form and applying a mathematical algorithm to classify the results into a classification group, i.e., an mGluR5 responder.

Typically the methods described above include control samples such as samples that are fully methylated and samples that are partially methylated. DNA purified from fragile X patient's B-lymphocytes (Camden, N.J.) can be used to generate appropriate controls or clinical samples which are already determined to have a particular methylation status can be used. Typically the methods described above include control samples. Samples taken from individuals that are fully methylated (or more than 95% methylated) can serve as positive controls and samples that are partially methylated can serve as negative controls. Such samples are readily available in the art or can be commercially purchased from, e.g., ATCC (American Type Culture Collection (ATCC), The National Institute for Biological Standards and Control (NIBSC) or Coriell institute for medical research. In one example, the positive control can be a fully methylated sample from NIBSC (07/170; Hertfordshire, England) and the negative control can be a partially methylated sample from NIBSC (07/174; Hertfordshire, England). The control can either be run simultaneously with the test sample or can be represented as a predetermined value based on the technology used to determine the methylation status of the sample. In one example, the predetermined value is a Delta Ct value which is obtained using quantitative PCR (as described herein).

The oligonucleotides of the invention also include variants of the sequences or sequences that are substantially similar to the oligonucleotides of the invention. Variants include sequences that are altered by one or more bases, such as 2, 3, 4, 5, 6, 7, 8, 9 or 10 but can still anneal to the specific locations on the FMR1 promoter sequence of interest. The term “substantially” when used in relation to annealing or hybridisation, means that the oligonucleotide or probe nucleic acid sequence should be sufficiently complementary to hybridise or anneal to its respective nucleic acid. As used herein, the term “hybridisation” refers to the process by which a strand of nucleic acid joins with a complementary strand. In one example, the oligonucleotide is between 14-30 bases. In another example, the oligonucleotide is between 18-30 bases and includes the sequence of SEQ ID NO:4, SEQ ID NO:5 or SEQ ID NO: 6, or variants thereof.

Oligonucleotides may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

FMR1 mRNA Determination

FMR1 mRNA levels can also be used as a predictive marker to determine if an individual is likely to be responsive to mGluR5. Samples from individuals having FXS who lack the presence of an FMR1 mRNA transcript or have a reduced level of FMR1 mRNA transcript compared to a control are determined to be mGluR5 responders. This determination can serve solely to classify an individual as being an mGluR5 responder or can be used with either, or both, FMR1 gene methylation status and FMR1 protein determinations as a means of complementing other assay results.

Levels of FMR1 mRNA are measured using any of a number of techniques known to those skilled in the art including, but not limited to Northern blot analysis, nuclease protection assays (NPA), in situ hybridization, reverse transcription-polymerase chain reaction (RT-PCR), RT-PCR ELISA, TaqMan-based quantitative RT-PCR (probe-based quantitative RT-PCR) and SYBR green-based quantitative RT-PCR.

Using the methods of the invention an individual is classified as an mGluR5 responder when no FMR1 mRNA is detected in the sample. Individuals who have reduced or low amounts of mGluR5 mRNA transcript are also an mGluR5 responder, e.g., a sample that has only 50% (4, 30, 20, 10, 9, 8, 7, 6, 5, 4, 3, 2, or 1%) of the FMR1 mRNA transcript compared to a control (healthy individual).

In one example, detection of mRNA levels involves contacting the isolated mRNA with an oligonucleotide that can hybridize to the mRNA encoded by the FMR1 gene being detected. The nucleic acid probe can typically be, for example, a full-length cDNA, or a portion thereof, such as an oligonucleotide of at least 7, 15, 30, 50, or 100 nucleotides in length and sufficient to specifically hybridize under stringent conditions to the mRNA. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose.

In another example, the level of the FMR1 mRNA can be determined by reverse transcription-polymerase chain reaction (RT-PCR), RT-PCR ELISA, TaqMan-based quantitative RT-PCR (probe-based quantitative RT-PCR) and SYBR green-based quantitative RT-PCR. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

### Protein

FMR1 protein levels can also be used as a predictive marker to determine if an individual is likely to be responsive to mGluR5. Samples from individuals having FXS who lack the presence of an FMR1 protein, or have a reduced amount of FMR1 protein as compared to a control, are determined to be mGluR5 responders. This determination can serve solely to classify an individual as being an mGluR5 responder or can be used with either, or both, FMR1 gene methylation status and FMR1 mRNA determinations as a means of complementing other assay results.

The detection of the FMR1 protein can be performed using any known method in the art including but not limited to immunocytochemical staining, ELISA, flow cytometry, Western blot, immunohistochemistry, spectrophotometry, HPLC, mass spectrometry and time-resolved Forster resonance energy transfer (TR-FRET).

Using the methods of the invention an individual is classified as an mGluR5 responder when no FMR1 protein is detected in the sample. Individuals who have reduced or low amounts of mGluR5 protein are also mGluR5 responders, e.g., a sample that has only 50% (40, 30, 20, 10, 9, 8, 7, 6, 5, 4, 3, 2, or 1%) of the amount of the FMR1 protein in the sample as compared to a control (healthy individual).

One method for detecting the FMR1 protein in a sample is by means of a binding protein capable of interacting specifically with a marker protein. Preferably, labeled antibodies, binding portions thereof, or other FMR1 binding partners can be used. The antibodies can be monoclonal or polyclonal in origin, or may be biosynthetically produced. The FMR1 binding partners may also be naturally occurring molecules or synthetically produced. The amount of complexed FMR1 protein, e.g., the amount of FMR1 protein associated with the binding protein, is determined using standard protein detection methodologies described in the art. A detailed review of immunological assay design, theory and protocols can be found in numerous texts in the art, including Practical Immunology, Butt, W. R., ed., Marcel Dekker, New York, 1984.

A variety of assays are available for detecting proteins with labeled antibodies. In a one-step assay, the FMR1 molecule, if it is present, is immobilized and incubated with a labeled antibody. The labeled antibody binds to the immobilized target molecule. After washing to remove unbound molecules, the sample is assayed for the presence of the label.

In a two-step assay, immobilized FMR1 molecule is incubated with an unlabeled antibody. The FMR1-unlabeled antibody complex, if present, is then bound to a second, labeled antibody that is specific for the unlabeled antibody. The sample is washed and assayed for the presence of the label.

The choice of marker used to label the antibodies will vary depending upon the application. However, the choice of the marker is readily determinable to one skilled in the art.

The antibodies may be labeled with a radioactive atom, an enzyme, a chromophoric or fluorescent moiety, or a colorimetric tag. The choice of tagging label also will depend on the detection limitations desired. Enzyme assays (ELISAs) typically allow detection of a colored product formed by interaction of the enzyme-tagged complex with an enzyme substrate. Some examples of radioactive atoms include ³²P, ¹²⁵I, ³H, and ¹⁴P. Some examples of enzymes include horseradish peroxidase, alkaline phosphatase, beta-galactosidase, and glucose-6-phosphate dehydrogenase. Some examples of chromophoric moieties include fluorescein and rhodamine. The antibodies may be conjugated to these labels by methods known in the art. For example, enzymes and chromophoric molecules may be conjugated to the antibodies by means of coupling agents, such as dialdehydes, carbodiimides, dimaleimides, and the like. Alternatively, conjugation may occur through a ligand-receptor pair. Some suitable ligand-receptor pairs include, for example, biotin-avidin or -streptavidin, and antibody-antigen.

In one aspect, the present invention contemplates the use of a sandwich technique for detecting FMR1 proteins in serum and other biological fluids. The technique requires two antibodies capable of binding the protein of interest: e.g., one immobilized onto a solid support, and one free in solution, but labeled with some easily detectable chemical compound. Examples of chemical labels that may be used for the second antibody include but are not limited to radioisotopes, fluorescent compounds, and enzymes or other molecules which generate colored or electrochemically active products when exposed to a reactant or enzyme substrate. When samples containing the FMR1 protein are placed in this system, the FMR1 protein binds to both the immobilized antibody and the labeled antibody. The result is a “sandwich” immune complex on the support's surface. The complexed protein is detected by washing away nonbound sample components and excess labeled antibody, and measuring the amount of labeled antibody complexed to protein on the support's surface. The sandwich immunoassay is highly specific and very sensitive, provided that labels with good limits of detection are used.

Preferably, the presence of FMR1 in a sample is detected by radioimmunoassays or enzyme-linked immunoassays, competitive binding enzyme-linked immunoassays, dot blot, Western blot, chromatography, preferably high performance liquid chromatography (HPLC), or other assays known in the art.

Dot blotting is routinely practiced by the skilled artisan to detect a desired protein using an antibody as a probe (Promega Protocols and Applications Guide, Second Edition, 1991, Page 263, Promega Corporation). Samples are applied to a membrane using a dot blot apparatus. A labeled probe is incubated with the membrane, and the presence of the protein is detected.

Western blot analysis is well known to the skilled artisan (Sambrook et al., Molecular Cloning, A Laboratory Manual, 1989, Vol. 3, Chapter 18, Cold Spring Harbor Laboratory). In Western blot, the sample is separated by SDS-PAGE. The gel is transferred to a membrane. The membrane is incubated with labeled antibody for detection of the desired protein.

The assays described above involve steps such as but not limited to, immunoblotting, immunodiffusion, immunoelectrophoresis, or immunoprecipitation.

In another example, the presence of FMRP in a sample is detected using time-resolved resonance energy transfer (TR-FRET). TR-FRET has been used to detect a number of different molecules including cAMP (Gabriel et al, 2003, Assay Drug Dev Technol. 1, 291-303) and mutated polyQ (WO 2010/015592). In one example the method includes contacting a biological sample with a first FMRP specific antibody, which is labeled with a lanthanoide ion cryptate (such as europium or terbium cryptate), and a second FMRP specific antibody labeled with an appropriate fluorogenic molecule such as XL-665 (a phycobilliprotein hetero-hexameric structure of 105 kDa available from CisBio) or a D2 acceptor. In the present method the antibodies are selected such that when they bind FMRP the lanthanide-cryptate emits energy which results in a proximity-dependent, time resolved FRET emission of the fluorophore. The amount of FMRP in the sample is quantified by measuring the fluorescence emitted from the fluorophore. Any FMRP specific antibodies can be used such as F4055 (Sigma, RTGKDRNQKKEKPDSVDG; SEQ ID NO:7); 2160 (millipore; ITVAFENNWQPDRQIPFHD; SEQ ID NO:8) and H00002332-M03 (Abnova; ATKDTFHKIKLDVPEDLRQMCAKEAAHKDFKKAVGAF SVTYDPENYQLVI; SEQ ID NO:9).

### Diagnostic and Prognostic Assays

The methods described herein can be utilized as a diagnostic assay to identify those subjects having Fragile X Syndrome who are likely to respond to an mGluR5 antagonist or can be used as a prognostic assay to identify subjects who are at risk of developing Fragile X Syndrome and who would benefit from receiving an mGluR5 antagonist. Prognostic assays can be used for predictive purposes or prophylactic purposes to treat an individual who is at risk of developing FXS.

The method of the invention can also be used not just for individuals identified to have FXS but for any individual who exhibits a CGG repeat length expansion in the FMR1 gene promoter, e.g., above 55 repeats. It is envisioned that such a population will benefit from mGluR5 treatment. Thus, the present invention provides a method in which a test sample is obtained from a subject who exhibits a CGG repeat length expansion in the FMR1 gene promoter, and the silencing of the FMR1 gene is determined, e.g., by determining the methylation status of the FMR1 promoter, detecting for the presence of the FMR1 protein and/or FMR1 mRNA. The presence of any, or any combination, of the following: if all, or predominately all of the FMR1 gene region is methylated, lack of FMR1 protein or mRNA in the sample, lack of FMR1 protein or lack of mRNA in the sample, is indicative of a subject who is an mGluR5 responder.

The method of the invention can be used as a prognostic assay to determine whether a subject should be administered an mGluR5 antagonist so as to prevent the onset of Fragile X syndrome or to reduce the severity of Fragile X syndrome. In one example, an individual can be determined to be at risk of developing FXS using any of the standard methods known in the art such as detecting CGG repeats or evaluating the family history of that individual. Once an individual has been determined to be at risk of FXS, that individual is further evaluated for the presence of any one or more of the following biomarkers: if all, or predominately all of the FMR1 gene region is methylated, lack of FMR1 protein or mRNA in the sample. The presence of one or more of the biomarkers described herein can be used to indicate that that individual should be administered an mGluR5 antagonist so as to prevent the onset of Fragile X syndrome or to reduce the severity of Fragile X syndrome. In one example, newborn infants determined to be at risk of developing FXS should be monitored for the presence of one or more of the biomarkers described herein so as to prevent the onset of Fragile X syndrome or to reduce the severity of Fragile X syndrome. The use of the present method to intervene early will maximize the therapeutic benefits of mGluR5.

The prognostic assay described herein can also be used in any individual who exhibits CGG repeat length expansion in the FMR1 gene. If an individual is determined based on the methods described herein to be an individual who will respond clinically to an mGluR5 antagonist, an mGluR5 antagonist will be administered to the individual. In general, a daily dosage in the range from about 5 to 1500 mg, preferably about 10 to about 1000 mg of the compound is conveniently administered to an individual having FXS. In one example, a daily dosage of 10 mg, 25 mg or 100 mg will be administered to the individual having FXS.

### Kits

The invention also encompasses kits for detecting the status of methylation of the FMR1 gene region, FMR1 mRNA expression or FMR1 protein levels in a biological sample (a test sample). Such kits can be used to determine if a subject having FXS is likely to respond to treatment with an mGluR5 antagonist. For example, the kit can comprise a labeled compound or agent capable of detecting the FMR1 protein or mRNA in a biological sample and means for determining the amount of an FMR1 protein (e.g., an anti-FMR1 antibody or an oligonucleotide probe that binds to DNA encoding an FMR1 protein) or mRNA transcript in the biological sample. The kit can also include primers that can be used to determine the extent of methylation of the FMR1 gene region as discussed above. Furthermore the kit can include appropriate control samples.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for it's use.

The following non-limiting Examples illustrate the invention.

### EXAMPLES

### Example 1

### Study set up to identify if a subset of patients exist that respond to (−)-(3aR,4S,7aR)-4-Hydroxy-4-m-tolylethynyl-octahydro-indole-1-carboxylic acid methyl ester treatment

The present examples were performed as a follow up to a clinical trial which investigated whether the mGluR5 antagonist (−)-(3aR,4S,7aR)-4-Hydroxy-4-m-tolylethynyl-octahydro-indole-1-carboxylic acid methyl ester could provide beneficial treatment to a subject having FXS. The present study was set up to identify if a subset of patients exist that respond to (−)-(3aR,4S,7aR)-4-Hydroxy-4-m-tolylethynyl-octahydro-indole-1-carboxylic acid methyl ester treatment. In an attempt to identify such a subset of patients who respond to (−)-(3aR,4S,7aR)-4-Hydroxy-4-m-tolylethynyl-octahydro-indole-1-carboxylic acid methyl ester treatment, a study was conducted to investigate the relationship between FMR1 methylation/mRNA expression and (−)-(3aR,4S,7aR)-4-Hydroxy-4-m-tolylethynyl-octahydro-indole-1-carboxylic acid methyl ester efficacy in the study.

### Clinical Samples:

Among the 30 patients who completed the clinical study, 26 consented for pharmacogenetic/pharmacogenomic assessments. Genomic DNA was extracted from whole blood according to the instructions from Gentra (Minneapolis, Minn.). Total RNA was extracted from whole blood according to the instructions from Qiagen (Valencia, Calif.). A total of 26 DNA and 24 RNA samples were successfully extracted and analyzed. In addition, normal control DNAs were purchased from Coriell Institute (Camden, N.J.).

### Methylation-Specific PCR (MSP) Assay:

Genomic DNA was treated with bisulfite (Qiagen, Valencia, Calif.). MSP was performed using the CpG WIZ Fragile X Amplification Kit from Chemicon (Temecula, Calif.) according to manufacture's instruction. A total of 26 patients and 4 normal controls were analyzed.

Quantitative RT-PCR (qRT-PCR) Assay:

FMR1 mRNA expression was measured by Taqman real-time PCR. The primers and probe were designed by Applied Biosystems (Foster City, Calif.; fragile X mental retardation 1 Hs 00924544_ml, Glyceraldehyde-3-phosphate dehydrogenase Hs 99999905_ml; ubiquitin C Hs 00824723_ml)

The expression levels of the control genes, GAPDH and UBC, were used to adjust for inter-sample variability. The data is presented as normalized Ct (cycle threshold). A Ct value≧36 is considered to be the background of the assay. A total of 24 patients and 9 normal controls were analyzed.

### Bisulfate-Sequencing:

Genomic DNA was treated with bisulfate (Qiagen, Valencia, Calif.). The following primers were used to amplify the FMR1 promoter with 22 CpG sites: 5′-GTTATTGAGTGTATTTTTGTAGAAATGGG-3′ (SEQ ID NO:10); and 5′-CCCTCTCTCTTCAAATAACCTAAAAAC-3′ (SEQ ID NO:11). The 196-bp FMR1 promoter was cloned using the TA cloning kit from Invitrogen (Carlsbad, Calif.) and 7-13 clones per patient were sequenced using ABI3730XL (Foster City, Calif.). More details can be found in the BMD report. A total of 26 patients and 4 normal controls were analyzed.

### Results:

The initial MSP analysis showed that 8 patients were fully methylated and 18 patients were partially methylated. The fully methylated patients did not express the FMR1 mRNA while the partially methylated patients expressed various levels of the mRNA. The data suggest a strong correlation between the FMR1 methylation and transcription in peripheral blood. More importantly, the patients who were fully methylated or did not express the FMR1 mRNA showed significantly greater improvement in response to (−)-(3aR,4S,7aR)-4-Hydroxy-4-m-tolylethynyl-octahydro-indole-1-carboxylic acid methyl ester over placebo at day 19 (ABC-C: p<0.001, CGI-efficacy index: p<0.001) (Table 1-4). In contrast, there were no significant differences between (−)-(3aR,4S,7aR)-4-Hydroxy-4-m-tolylethynyl-octahydro-indole-1-carboxylic acid methyl ester and placebo in those who were partially methylated or expressed the FMR1 mRNA.

To gain more information on the methylation pattern and verify the MSP assay, bisulfite-sequencing for the FMR1 promoter with 22 CpG sites was performed. The bisulfite-sequencing data was consistent with the MSP data for all patients except three discrepancies which may have occurred due to, at least in part, limited number of clones per patient that were sequenced. To further evaluate the impact of FMR1 methylation on (−)-(3aR,4S,7aR)-4-Hydroxy-4-m-tolylethynyl-octahydro-indole-1-carboxylic acid methyl ester efficacy, the methylation status of the three patients with discrepancies were reclassified by aggregate consideration of the initial MSP, bisulfite-sequencing, and additional MSP data. Two of the three patients could be reasonably reclassified. The remaining one could not be reclassified based on the current data and was removed from the efficacy analysis. The new statistical analysis showed that (−)-(3aR,4S,7aR)-4-Hydroxy-4-m-tolylethynyl-octahydro-indole-1-carboxylic acid methyl ester response remained significantly higher than placebo in the fully methylated patients (ABC-C: p<0.001, CGI-efficacy index: p<0.001) (Table 5-6).

Taken together, the overall data indicate that the FMR1 methylation or mRNA expression could serve as a predictive biomarker for the clinical response in FXS patients treated with (−)-(3aR,4S, 7aR)-4-Hydroxy-4-m-tolylethynyl-octahydro-indole-1-carboxylic acid methyl ester.

TABLE 1
ABC-C results by FMR1 methylation status

| Study | | | Treatment | Lower | Upper | |
|---|---|---|---|---|---|---|
| population | Day | Efficacy variable | difference* | 90% CI | 90% CI | P value** |
| Patients | 19 | Total of subscales | −30.85 | −41.37 | −20.32 | <0.001 |
| with full | | Subscale I (Irritability) | −4.33 | −7.28 | −1.39 | 0.020 |
| methylation | | Subscale II (Lethargy) | −6.55 | −10.75 | −2.35 | 0.014 |
| (n = 8) | | Subscale III | −3.96 | −6.92 | −1.01 | 0.032 |
| | | (Stereotype) | | | | |
| | | Subscale IV | −8.77 | −11.71 | −5.82 | <0.001 |
| | | (Hyperactivity) | | | | |
| | | Subscale V | −4.48 | −6.20 | −2.75 | <0.001 |
| | | (Inappropriate Speech) | | | | |
| Patients | 19 | Total of subscales | 5.66 | −2.33 | 13.65 | 0.241 |
| with partial | | Subscale I (Irritability) | −0.67 | −2.67 | 1.34 | 0.580 |
| methylation | | Subscale II (Lethargy) | 3.76 | 0.41 | 7.11 | 0.066 |
| (n = 18) | | Subscale III | 0.89 | −0.58 | 2.35 | 0.315 |
| | | (Stereotype) | | | | |
| | | Subscale IV | 0.42 | −2.13 | 2.98 | 0.782 |
| | | (Hyperactivity) | | | | |
| | | Subscale V | 1.09 | −0.49 | 2.67 | 0.253 |
| | | (Inappropriate Speech) | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in ABC-C scores indicates an improvement.

TABLE 2
CGI results by FMR1 methylation status

| Study | | | Treatment | Lower | Upper | |
|---|---|---|---|---|---|---|
| population | Day | Efficacy variable | difference* | 90% CI | 90% CI | P value** |
| Patients | 19 | CGI-global | −1.94 | −2.45 | −1.44 | <0.001 |
| with full | | improvement | | | | |
| methylation | | CGI-efficacy index | 1.86 | 1.31 | 2.42 | <0.001 |
| (n = 8) | | | | | | |
| Patients | 19 | CGI-global | 0.67 | 0.15 | 1.18 | 0.035 |
| with partial | | improvement | | | | |
| methylation | | CGI-efficacy index | −0.58 | −1.10 | −0.07 | 0.064 |
| (n = 18) | | | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in CGI-global improvement indicates an improvement.
An increase in CGI-efficacy index indicates an improvement.

TABLE 3
ABC-C results by FMR1 mRMA expression

| Study | | | Treatment | Lower | Upper | |
|---|---|---|---|---|---|---|
| population | Day | Efficacy variable | difference* | 90% CI | 90% CI | P value** |
| Patients who | 19 | Total of subscales | −32.19 | −45.00 | −19.38 | <0.001 |
| did not | | Subscale I (Irritability) | −3.69 | −7.26 | −0.13 | 0.089 |
| express | | Subscale II (Lethargy) | −6.71 | −12.03 | −1.39 | 0.042 |
| FMR1 mRNA | | Subscale III | −3.03 | −6.70 | 0.64 | 0.167 |
| (n = 7) | | (Stereotype) | | | | |
| | | Subscale IV | −9.12 | −12.80 | −5.44 | <0.001 |
| | | (Hyperactivity) | | | | |
| | | Subscale V | −4.66 | −6.84 | −2.48 | 0.005 |
| | | (Inappropriate Speech) | | | | |
| Patients who | 19 | Total of subscales | 2.23 | −5.39 | 9.85 | 0.627 |
| expressed | | Subscale I (Irritability) | −0.81 | −2.86 | 1.25 | 0.513 |
| FMR1 mRNA | | Subscale II (Lethargy) | 1.93 | −1.07 | 4.93 | 0.287 |
| (n = 17) | | Subscale III | 0.32 | −1.08 | 1.73 | 0.703 |
| | | (Stereotype) | | | | |
| | | Subscale IV | −0.16 | −2.71 | 2.39 | 0.916 |
| | | (Hyperactivity) | | | | |
| | | Subscale V | 1.09 | −0.59 | 2.77 | 0.280 |
| | | (Inappropriate Speech) | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in ABC-C scores indicates an improvement.

TABLE 4
CGI results by FMR1 mRMA expression

| Study | | | Treatment | Lower | Upper | |
|---|---|---|---|---|---|---|
| population | Day | Efficacy variable | difference* | 90% CI | 90% CI | P value** |
| Patients who | 19 | CGI-global | −2.02 | −2.64 | −1.41 | <0.001 |
| did not express | | improvement | | | | |
| FMR1 mRNA | | CGI-efficacy index | 1.99 | 1.30 | 2.68 | <0.001 |
| (n = 7) | | | | | | |
| Patients who | 19 | CGI-global | 0.56 | 0.02 | 1.09 | 0.087 |
| expressed | | improvement | | | | |
| FMR1 mRNA | | CGI-efficacy index | −0.47 | −1.00 | −0.06 | 0.147 |
| (n = 17) | | | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in CGI-global improvement indicates an improvement.
An increase in CGI-efficacy index indicates an improvement.

TABLE 5
ABC-C results by FMR1 methylation status (reclassified)

| Study | | | Treatment | Lower | Upper | |
|---|---|---|---|---|---|---|
| population | Day | Efficacy variable | difference* | 90% CI | 90% CI | P value** |
| Patients | 19 | Total of subscales | −27.82 | −39.05 | −16.59 | <0.001 |
| with full | | Subscale I (Irritability) | −2.66 | −5.37 | 0.05 | 0.106 |
| methylation | | Subscale II (Lethargy) | −5.53 | −10.87 | −0.18 | 0.090 |
| (determined by | | Subscale III | −5.06 | −8.66 | −1.46 | 0.027 |
| MSP and | | (Stereotype) | | | | |
| sequencing) | | Subscale IV | −8.55 | −12.27 | −4.84 | <0.001 |
| (n = 7) | | (Hyperactivity) | | | | |
| | | Subscale V | −4.31 | −6.26 | −2.36 | 0.001 |
| | | (Inappropriate Speech) | | | | |
| Patients | 19 | Total of subscales | 3.10 | −5.61 | 11.82 | 0.554 |
| with partial | | Subscale I (Irritability) | −1.15 | −3.45 | 1.16 | 0.410 |
| methylation | | Subscale II (Lethargy) | 2.66 | −0.81 | 6.13 | 0.206 |
| (determined by | | Subscale III | 0.78 | −0.70 | 2.25 | 0.383 |
| MSP and | | (Stereotype) | | | | |
| sequencing) | | Subscale IV | −0.21 | −2.85 | 2.43 | 0.894 |
| (n = 18) | | (Hyperactivity) | | | | |
| | | Subscale V | 0.81 | −0.80 | 2.41 | 0.403 |
| | | (Inappropriate Speech) | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in ABC-C scores indicates an improvement.

TABLE 6
CGI results by FMR1 methylation status (reclassified)

| Study | | | Treatment | Lower | Upper | |
|---|---|---|---|---|---|---|
| population | Day | Efficacy variable | difference* | 90% CI | 90% CI | P value** |
| Patients with full | 19 | CGI-global | −1.78 | −2.34 | −1.22 | <0.001 |
| methylation (by MSP | | improvement | | | | |
| and sequencing) | | CGI-efficacy index | 1.76 | 1.13 | 2.39 | <0.001 |
| (n = 7) | | | | | | |
| Patients with partial | 19 | CGI-global | 0.58 | 0.04 | 1.11 | 0.079 |
| methylation (by MSP | | improvement | | | | |
| and sequencing) | | CGI-efficacy index | −0.43 | −0.96 | 0.11 | 0.193 |
| (n = 18) | | | | | | |

*(−)-(3aR,4S,7aR)-4-Hydroxy-4-m-tolylethynyl-octahydro-indole-1-carboxylic acid methyl ester versus placebo. Adjusted for baseline values.
**Mixed model.
A decrease in CGI-global improvement indicates an improvement.
An increase in CGI-efficacy index indicates an improvement.

### Example 2

### Taqman Probe-Based Real-Time PCR Assay Post Restriction Enzyme Digestions for determination of FMR1 promoter methylation

### Clinical Samples:

Among the 26 genomic DNA purified from patients consented for pharmacogenetic/pharmacogenomic assessments in Example 1, twelve had sufficient amount to be analyzed by a probe-based methylation assay.

### Probe-Based Methylation Assay:

The assay is based on MethylScreen technology from Orion Genomics (St. Louis, Mo.). However, it combines MethylScreen's restriction enzyme DNA treatment with a Taqman hydrolysis probe-based real-time PCR. Briefly, in order to assess the methylation status of FMR1 promoter region, purified genomic DNA from EDTA-anticoagulant blood is digested with McrBC and HhaI, both from NEB (Ipswich, MA), independently and also congruently per manufacturer's instruction, leading to the following 4 conditions: 1) No enzyme digest; 2) McrBC digest; 3) HhaI digest; and 4) McrBC and HhaI double digest, all incubated at 37° C. for 16 hours before inactivation at 65° C. for 20 minutes. The restriction enzyme McrBC is methylation-dependent which means that it only cleaves the methylated DNA, and the restriction enzyme HhaI is methylation-sensitive which means it only cleaves the unmethylated DNA. Each condition contains the same amount of input DNA as 400 ng for real-time PCR detection, therefore by comparing each digest condition with no enzyme digest control, the remaining amount of amplifiable DNA after enzyme digestion could be quantitated by real-time PCR using primer pairs (Forward 5′-tgcagaaatgggcgttct (SEQ ID NO:4; Reverse 5′-gtgccgggtcgaaagac (SEQ ID NO:5)) and a FAM-labeled probe (Probe 5′ FAM-ctgaagggcggtgacaggtcg-BHQ1; (SEQ ID NO:6)). Prior to real-time PCR detection, the enzyme digestion mixes are subject to the treatment of a restriction enzyme AluI for 1 hour at 37° C. followed by 65° C. inactivation for 20 minutes. The PCR amplicon covers 15 CpG islands in the region of interest. The difference in digest condition and the control is reflected in a change in PCR cycle threshold (delta Ct). The Clinical Cut-off Region constitutes a dCt (difference in PCR Cycle threshold values between McrBC and Untreated channels) range of ˜5 to 14. This correlates with a methylation percent range of between 94-100% CTA. Through calculation per an algorithm, a methylation percent in FMR1 promoter region could be determined from the sample DNA.

### Results:

Out of 12 patients in the original proof-of-concept study described in Example 1, the probe-based methylation analysis showed that 3 patients were fully methylated and 9 patients partially methylated, generating similar results as the MSP assay. Patients that were fully methylated had a Delta Ct ranging from between −9.95 to −10.27 and had a percentage methylation ranging between 99.9-99.92. Patients that were partially methylated had a Delta Ct ranging between −1.77 to −7.29 and a percentage methylation ranging between 70.68 to 99.36. The fully methylated patients did not express the FMR1 mRNA while the partially methylated patients expressed various levels of the mRNA. The data suggest a strong correlation between the FMR1 methylation and transcription in peripheral blood. More importantly, the patients who were fully methylated and/or did not express the FMR1 mRNA showed significantly greater improvement in response to (−)-(3aR,4S,7aR)-4-Hydroxy-4-m-tolylethynyl-octahydro-indole-1-carboxylic acid methyl ester over placebo at day 19 (ABC-C: p<0.001, CGI-efficacy index: p<0.001). In contrast, there were no significant differences between (−)-(3aR,4S,7aR)-4-Hydroxy-4-m-tolylethynyl-octahydro-indole-1-carboxylic acid methyl ester and placebo in those who were partially methylated or expressed the FMR1 mRNA.

### Example 3

### FMRP determination using Time-Resolved Forster Resonance Energy Transfer (TR-FRET) Immunoassay

The following antibodies were used in the TR-FRET immunoassays: F4055 (Sigma, RTGKDRNQKKEKPDSVDG (SEQ ID NO:7)); 2160 (millipore; ITVAFENNWQPDRQIPFHD; (SEQ ID NO:8) and H00002332-M03 (Abnova; ATKDTFHKIKLDVPEDLRQMCAKEAAHKDFKKAVGAFSVTYDPENYQLVI; (SEQ ID NO:9).

### Temperature Dependent Signal Kinetics for Human FMRP Protein Detection by F4055-H00002332-M03 Antibody Combination

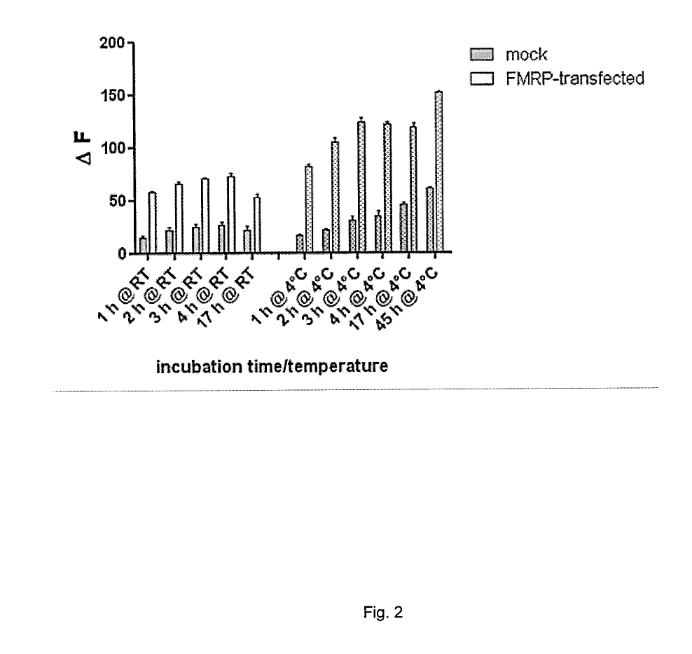

HEK293T cells were transiently transfected with eGFP plasmid (mock) or human FMRP plasmid (FMRP-transfected). Cells were lysed in M-PER (Pierce) lyis buffer, 150 nM NaCl and Protease Inhibitor. 1 μg total protein in 5 μl were loaded per low-volume 384-well and 1 μl of antibody detection buffer. Results shown in FIG. 2.

### Temperature Dependent Signal Kinetics for Human FMRP Protein Detection by MAB2160-F4055 Antibody Combination

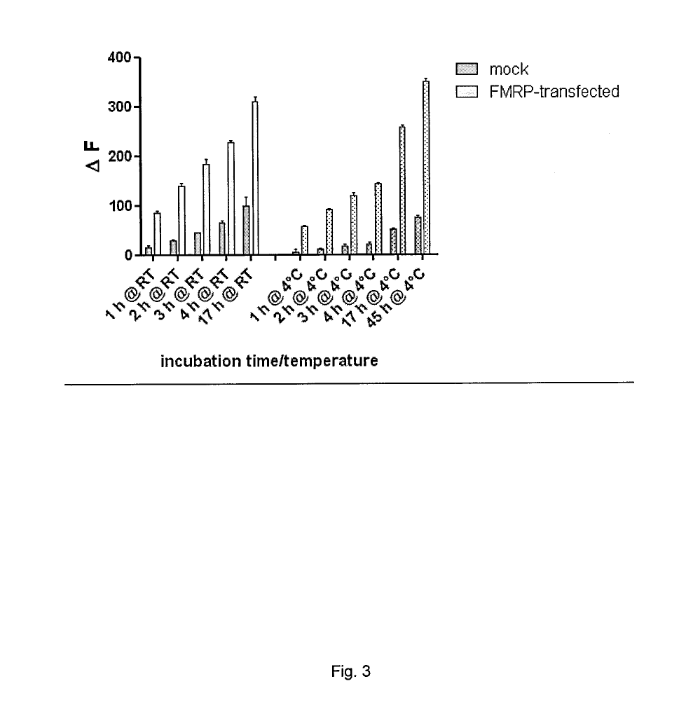

HEK293T cells were transiently transfected with eGFP plasmid (mock) or human FMRP plasmid (FRMP-transfected). Cells were lysed in M-PER (Pierce) lyis buffer, 150 nM NaCl and Protease Inhibitor. 1 μg total protein in 5 μl were loaded per low-volume 384-well and 1 μl of antibody detection buffer (final antibody amount: 0.6 ng/well Millipore MAB2160-Tb & 20 ng/well Sigma F4055-d2). Results shown in FIG. 3.

### Endogenous Human FMRP Protein Detection Primary Human Fibroblasts

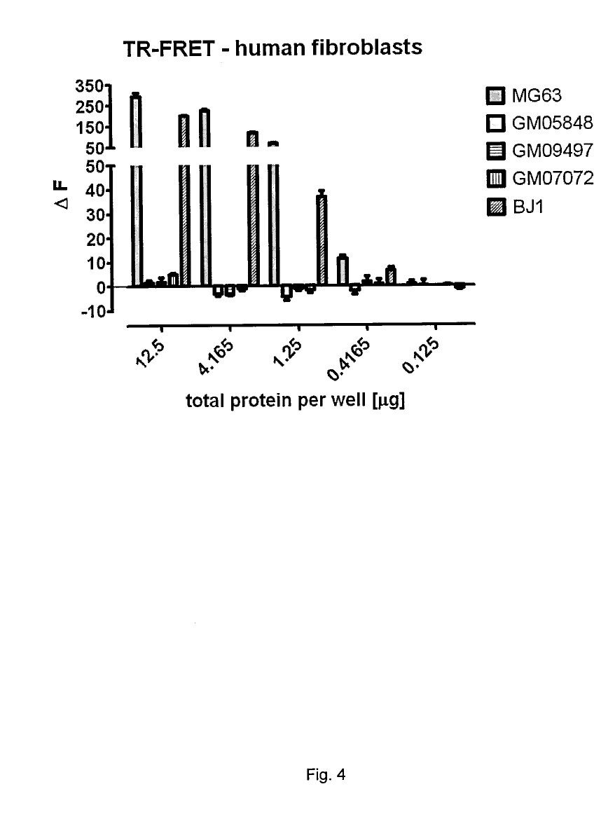

Healthy control fibroblasts (BJ1 and MG63) or fully methylated fragile X patient fibroblasts (GM05848B, GM09497A and GM07072) were lysed in M-PER (Pierce) lyis buffer, 150 nM NaCl and Protease Inhibitor. Total protein concentration was adjusted to 12.5 μg total protein per 5 μl (approx. 8 000 cells/1 μl). Protein concentration dilution for each lysate was loaded per low-volume 384-well and 1 μl of antibody detection buffer (final antibody amount: 0.3 ng/well Millipore MAB2160-Tb & 3 ng/well Abnova H00002332-M03-d2). Results shown in FIG. 4.

What is claimed is:

1. A method of determining the responsiveness of an individual with Fragile X Syndrome (FXS) to treatment with an mGluR5 antagonist, the method comprising:
isolating an RNA sample from an individual having Fragile X Syndrome;
performing an assay which detects an FMR1 mRNA transcript in the RNA sample, and
assigning the individual as an mGluR5 responder if the sample has a reduced level of FMR1 mRNA expression compared to a control.

2. The method of claim 1, wherein the assay is selected from the group consisting of Northern blot analysis, reverse transcription-polymerase chain reaction (RT-PCR), RT-PCR ELISA, TaqMan-based quantitative RT-PCR (probe-based quantitative RT-PCR) and SYBR green-based quantitative RT-PCR.

3. A method of determining the responsiveness of an individual with FXS to treatment with an mGluR5 antagonist, the method comprising:
isolating a sample from an individual having Fragile X Syndrome;
performing an assay which determines the amount of FMR1 protein in the sample; and
assigning the individual as an mGluR5 responder if the sample has a reduced amount of FMR1 protein (FMRP) compared to a control.

4. The method of claim 3, wherein the assay is selected from the group consisting of immunohistochemistry, ELISA, flow cytometry, Western blot, HPLC, and mass spectrometry.

5. A method for determining responsiveness of an individual with Fragile X Syndrome (FXS) to treatment with an mGluR5 antagonist, the method comprising:
providing a nucleic acid sample from an individual having FXS;
determining the extent of methylation of a fragile X mental retardation 1 (FMR1) gene region in the sample, wherein the level of methylation in the sample relative to a control is indicative whether the individual is an mGluR5 responder

6. A method for determining responsiveness of an individual with Fragile X Syndrome (FXS) to treatment with an mGluR5 antagonist, the method comprising:
providing a nucleic acid sample from the individual having Fragile X Syndrome;
determining the extent of methylation of a fragile X mental retardation 1 (FMR1) gene region in the sample, and
assigning the individual as an mGluR5 responder if the FMR1 gene region present in the sample is fully methylated.

7. The method of claim 1 wherein the mGluR5 antagonist is (−)-(3aR,4S,7aR)-4-Hydroxy-4-m-tolylethynyl-octahydro-indole-1-carboxylic acid methyl ester.

8. The method of claim 6, wherein the determination can be performed using an assay selected from methylation-sensitive restriction enzyme digestion combined with at least one of: Southernblot or quantitative PCR (probe- or SYBR green-based) or from bisulfate DNA modification combined with at least one of: methylation specific PCR (MSP), quantitative methylation specific PCR (probe- or SYBR green-based) or pyrosequencing.

9. A method of determining the responsiveness of an individual with Fragile X Syndrome (FXS) to treatment with an mGluR5 antagonist, the method comprising:
determining in a sample from an individual having FXS for the presence of an FMR1 mRNA transcript, an FMR1 protein, or methylation of an FMR1 gene region, or any combination thereof; and
assigning the individual as an mGluR5 responder if the sample has a reduced level of FMR1 mRNA compared to a control, a reduced amount of FMR1 protein compared to a control, or if the FMR1 gene region present is fully methylated.

10. The method of claim 9, wherein the method comprises determining for the presence of FMR1 mRNA and FMR1 protein.

11. The method of claim 6, wherein the FMR1 gene region is SEQ ID NO: 1, SEQ ID NO:2 or SEQ ID NO:3.

12. The method of claim 1 wherein the method further comprises administering an mGluR5 antagonist.

13. The method of claim 12, wherein the mGluR5 antagonist is (−)-(3aR,4S,7aR)-4-Hydroxy-4-m-tolylethynyl-octahydro-indole-1-carboxylic acid methyl ester.

14. A diagnostic kit for determining if an individual with Fragile X Syndrome (FXS) is an mGluR5 antagonist responder comprising:
an agent for measuring an FMR1 mRNA transcript, FMR1 protein levels, or methylation of an FMR1 gene region, or any combination thereof;
and instructions for use.

15. The method of claim 3 wherein the mGluR5 antagonist is (−)-(3aR,4S,7aR)-4-Hydroxy-4-m-tolylethynyl-octahydro-indole-1-carboxylic acid methyl ester.

16. The method of claim 5 wherein the mGluR5 antagonist is (−)-(3aR,4S,7aR)-4-Hydroxy-4-m-tolylethynyl-octahydro-indole-1-carboxylic acid methyl ester.

17. The method of claim 6 wherein the mGluR5 antagonist is (−)-(3aR,4S,7aR)-4-Hydroxy-4-m-tolylethynyl-octahydro-indole-1-carboxylic acid methyl ester.

18. The method of claim 8, wherein the FMR1 gene region is SEQ ID NO: 1, SEQ ID NO:2 or SEQ ID NO:3.

19. The method of claim 9, wherein the FMR1 gene region is SEQ ID NO: 1, SEQ ID NO:2 or SEQ ID NO:3.

20. The method of claim 3 wherein the method further comprises administering an mGluR5 antagonist.

21. The method of claim 5 wherein the method further comprises administering an mGluR5 antagonist.

22. The method of claim 6 wherein the method further comprises administering an mGluR5 antagonist.

23. The method of claim 9 wherein the method further comprises administering an mGluR5 antagonist.


SEQUENCE LISTING (11 sequences)

SEQ ID NO: 1 - DNA, 1020 bp, Homo sapiens
tgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctctccgactccgagaggccctagcgcctatc

SEQ ID NO: 2 - DNA, 196 bp, Homo sapiens
gccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagaggg

SEQ ID NO: 3 - DNA, 120 bp, Homo sapiens
tgcagaaatgggcgttctggccctcgcgaggcagtgcgacctgtcaccgcccttcagccttcccgccctccaccaagcccgcgcacgcccggcccgcgcgtctgtctttcgacccggcac

SEQ ID NO: 4 - DNA, 18 bp, Homo sapiens
tgcagaaatgggcgttct

SEQ ID NO: 5 - DNA, 17 bp, Homo sapiens
gtgccgggtcgaaagac

SEQ ID NO: 6 - DNA, 21 bp, Homo sapiens
ctgaagggcggtgacaggtcg

SEQ ID NO: 7 - protein, 18 aa, Homo sapiens
RTGKDRNQKKEKPDSVDG

SEQ ID NO: 8 - protein, 19 aa, Homo sapiens
ITVAFENNWQPDRQIPFHD

SEQ ID NO: 9 - protein, 50 aa, Homo sapiens
ATKDTFHKIKLDVPEDLRQMCAKEAAHKDFKKAVGAFSVTYDPENYQLVI

SEQ ID NO: 10 - DNA, 29 bp, Homo sapiens
gttattgagtgtatttttgtagaaatggg

SEQ ID NO: 11 - DNA, 27 bp, Homo sapiens
ccctctctcttcaaataacctaaaaac